(19) United States
(12) Patent Application Publication
(10) Pub. No.: US 2016/0263196 A1
(43) Pub. Date: Sep. 15, 2016

(54) HEMATOPOIETIC RECOVERY FROM RADIATION INJURY

(71) Applicant: SLOAN-KETTERING INSTITUTE FOR CANCER RESEARCH, New York, NY (US)

(72) Inventors: Jarrod A. Dudakov, New York, NY (US); Marcel R.M. van den Brink, New York, NY (US); Enrico Velardi, New York, NY (US)

(21) Appl. No.: 15/033,178

(22) PCT Filed: Nov. 3, 2014
(86) PCT No.: PCT/US14/63701

§ 371 Date: Apr. 29, 2016

Related U.S. Application Data
(60) Provisional application No. 61/899,659, filed on Nov. 4, 2013.

(51) Int. Cl. A61K 38/22; A61K 38/24

(52) U.S. Cl. CPC A61K 38/22; A61K 38/24

(57) ABSTRACT

Described herein are methods for the treatment of radiation injury by administration of sex steroid inhibitory (SSI) agents.

### GOVERNMENT RIGHTS

This invention was made with government support under grant numbers R01-HL069929, R01-AI100288, R01AI080455, R01-AI101406, and P01-CA023766 awarded by the National Institutes of Health; and also HHSN272200900059C by the United States National Institute of Allergy and Infectious Diseases. The United States government has certain rights in the invention.

### BACKGROUND

Hematopoietic stem cells are responsible for the formation of new blood and immune cells. Radiation exposure results in deleterious effects to numerous vital systems in the body, which may result in death.

### SUMMARY OF INVENTION

The present invention encompasses the finding that inhibition of the activity of sex hormones correlates with hematopoietic recovery from radiation injury. Promoting hematopoietic recovery facilitates survival and restoration of health following exposure to an otherwise lethal dose of radiation.

In some embodiments, the invention provides methods for treatment of radiation injury comprising a step of administering an agent that reduces the activity of a sex hormone to a subject suffering from or susceptible to radiation injury.

In some embodiments, radiation injury comprises a reduction in the number of myeloid cells.

In some embodiments, radiation injury results in a reduction in circulating levels of lymphoid cells, hemoglobin, and/or hematocrit.

In some embodiments, radiation injury comprises a reduction the number of red blood cells.

In some embodiments, radiation injury results from exposure to a lethal dose of radiation. In some embodiments, radiation injury results from accidental exposure to ionizing radiation. In some embodiments, radiation injury results from exposure to ionizing radiation from a weapon.

In some embodiments, a sex steroid inhibitor (SSI) agent is administered to a subject contemporaneously with radiation exposure. In some embodiments, a SSI agent is administered subsequent to radiation exposure. In some embodiments, an SSI agent is administered at least 1, 6, 12, 24, 48, or 72 hours subsequent to radiation exposure.

In some embodiments, a SSI agent administered to a subject reduces the level of a sex hormone in circulation. In some embodiments, an SSI agent inhibits the synthesis of a sex steroid. In some embodiments, an SSI agent decreases the level of testosterone in circulation. In some embodiments, an SSI agent decreases the level of estrogen in circulation. In some embodiments, an agent inhibits activity of a sex hormone receptor. In some embodiments, an agent modulates activity at a leutinizing hormone releasing hormone (LHRH) receptor. In some embodiments, an agent is a LHRH antagonist. In some embodiments, an LHRH antagonist is selected from degarelix, abarelix, ganirelix, cetrorelix, and combinations thereof.

In some embodiments, an agent is an LHRH agonist. In some embodiments, an LHRH agonist is selected from Leuprolide, Buserelin, Nafarelin, Histrelin, Goserelin, and Deslorelin. In some embodiments, an agent is an androgen receptor antagonist.

In some embodiments, an agent is an estrogen receptor antagonist. In some embodiments, an agent is a selective estrogen receptor modulator (SERM).

In some embodiments, an SSI agent is administered systemically. In some embodiments, an SSI agent is administered locally. In some embodiments, an SSI agent is administered by a route selected from subcutaneous, intramuscular, intravenous, intracerebroventricular, intra-abdominal, and intraosseous. In some embodiments, an SSI agent is administered into the abdominal wall. In some embodiments, the agent is administered orally.

In some embodiments, the invention provides methods for treatment of radiation injury comprising a step of removing or ablating at least one of a testicle or ovary from a subject suffering from or susceptible to radiation injury.

In some embodiments, the invention provides methods for promoting hematopoietic recovery subsequent to radiation injury in a subject comprising a step of administering an agent that reduces the activity of a sex hormone. In some embodiments, hematopoietic recovery comprises protection of hematopoietic stem cells. In some embodiments, hematopoietic recovery comprises recovery of white blood cells. In some embodiments, hematopoietic recovery comprises recovery of lymphocytes. In some embodiments, hematopoietic recovery comprises recovery of myeloid cells.

In some embodiments, hematopoietic recovery comprises improvement in one or more complete blood count measures. In some embodiments, improvement in blood count measure comprises an increase in hemoglobin level, an increase in hematocrit level, an increase in red blood cell number, and combinations thereof. In some embodiments, hematopoietic recovery comprises an increase in bone marrow cellularity.

In some embodiments, the invention provides methods for promoting hematopoietic recovery subsequent to radiation injury that results from exposure to a lethal dose of radiation.

In some embodiments, the invention provides pharmaceutical compositions for use in the treatment of radiation injury comprising an agent that reduces the activity of a sex hormone and a pharmaceutically acceptable carrier.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The Drawing included herein, which is composed of the following Figures, is for illustration purposes only not for limitation.

FIG. 1 shows an androgen receptor (AR) mediated negative regulation of DLL4 expression by action of testosterone. A, Molecular profile of thymic stromal cells (CD45⁻) 7 days after Degarelix treatment. Average and standard deviation of 3 samples, each obtained from 3 thymi polled, is represented. mRNA expression is calculated as a relative expression referred to each untreated control. B, Schematic representation of DLL4 promoter with the androgen receptor elements (AREs) represented by yellow boxes labeled A, B, C, and D (bp). Plots were generated using VISTA tool: each “peaks and valleys” graph represents percent conservation between the aligned sequences. Regions of high conservation are colored according to the annotation as exons (dark blue), UTRs (light blue) or non-coding (pink). C, ARE matrix logo as annotated in the JASPAR database is represented (top) among with ARE sequences identified in DLL4 promoter. Yellow shadows represent the androgen receptor (AR) core sequence. D, DLL4 expression in cTEC c9 24h after treatment with DTH or MDV3100 (MDV). mRNA expression is represented as relative expression compared untreated control. E, AR binding in the promoter regions represented in B, 2 h after DTH and MDV3100 (MDV) treatment is represented as enrichment over control IgG sample.

FIG. 2 shows a luteinizing-hormone-releasing hormone (LHRH) antagonist triggered thymic regeneration within 7 days after treatment. C57BL/6 mice were treated with vehicle (white), LHRH-Ag (black) or LHRH-Ant (grey) and their testosterone levels and thymi were analyzed at different time points. A, Analysis of testosterone levels in 8- to 12-weeks old male mice. B, Total thymic cellularity 7, 14, and 28 days after treatment. C, Absolute numbers of DN (Double negative, CD4⁻CD8⁻), DP (double positive CD4⁺CD8⁺) and CD4⁺ and CD8⁺ single positive thymocytes. D, Absolute number of cTEC (UEA-1^{lo}), mTEC^{lo} (UEA-1^{hi} MHCII^{lo}) and mTEC^{hi} (UEA-1^{hu} MHCII^{hi}). All populations were gated on CD45⁻ EpCAM⁺. E, Absolute numbers of total thymic cellularity of 9 months old male mice 28 days after treatment. F, Analysis of total thymic cellularity of 8 to 10-weeks old and 9 months old female mice 28 days after LHRH-Ant treatment. G, Thymic endothelial cells (PDGF-Rα⁺) and fibroblasts (CD31⁺) were analyzed at different time points. H, Absolute numbers of DN, DP, CD4⁺ SP and CD8⁺ SP thymocytes of 9 months old male mice 28 days after LHRH-Ag treatment. I, Absolute number of cTEC (UEA-1^{lo}), mTEC^{lo} (UEA-1^{hi} MHCII^{lo}), mTEC^{hi} (UEA-1^{hi} MHCII^{hi}), endothelial cells (PDGF-Rα⁺) and fibroblasts (CD31⁺) of 9 months old male mice 28 days after LHRH-Ant treatment. Results are expressed as the combined mean±SEM of 5-8 mice for each group representing at least two independent experiments. */̂ (p≦0.05); **/̂̂ (p≦0.01), ***/AAA (p≦0.001), compared with vehicle (*) and LHRH-Ag treated mice (̂).

FIG. 3 shows sex steroid inhibition (SSI) increases Dll4 signaling in the thymus. A, Molecular profile of thymic stromal cells (CD45⁻) 7 days after LHRH-Ant treatment. mRNA relative expression referred to each untreated control (n=8). B, mRNA expression of Hes1 and Ptcra in CD45⁺ enriched thymocytes 7 days after LHRH-Ant treatment. mRNA relative expression referred to each untreated control (n=8). C, Mean fluorescence intensity (MFI) of CD25 expression in CD45⁺CD4⁻ CD8⁻ CD3⁻ CD25⁺ thymocytes 7 days after LHRH-Ant treatment. Results are expressed as the combined mean±SEM of 5-8 mice for each group representing at least two independent experiments. *(p≦0.05); **(p≦0.01), compared with vehicle control.

FIG. 4 shows SSI treatment restored thymopoiesis and accelerates peripheral reconstitution in immunocompromised recipients following sub-lethal-total body irradiation (SL-TBI). Young male C57Bl/6 mice were pretreated 5 days before SL-TBI with vehicle (black) or LHRH-Ant (grey) and their thymi and spleens were analyzed at different time points after irradiation. A, Absolute number of thymic cellularity. B, Absolute numbers of total splenocytes. C-D, Absolute numbers of total and naïve (CD4⁺N, CD62L^{hi} Cd44^{lo}) CD4⁺ T cells. E-F, Absolute number of total and naïve (CD8⁺N, CD62L^{hi}Cd44^{lo}) CD8⁺ T cells. G, CD5⁺ enriched splenocytes obtained from vehicle and LHRH-Ant treated mice 42 days after irradiation were cultured in vitro for proliferative experiments. H, LCMV viral titer in the spleen of mice treated with vehicle or LHRH-Ant before SL-TBI and infected at day 14 after irradiation. Viral titer was analyzed 8 days after infection. I-K, Lethally irradiated 8- to 12-weeks old male C57Bl/6 mice were pre-treated with vehicle (black) or LHRH-Ant (grey) treated and transplanted with 5×106 B10.BR TCD BM cells. I, Absolute number of thymic cellularity. J-K. Absolute number of total, effector memory (EF, CD62L^{lo} Cd44^{hi}), central memory (CM, CD62L^{hi}Cd44^{hi}) and naïve (CD62L^{lo} Cd44^{hi}) splenic cells 3 months after transplant. Results are expressed as the combined mean±SEM of 8-15 mice for each group representing at least two independent experiments. L, Absolute number of CD4⁺ SP, CD8⁺ SP, CD4⁺ CD8⁺ DP and CD4⁻ CD8⁻ DN thymocytes. M, Absolute numbers of cTEC (UEA-1^{lo}), mTEC^{lo} (UEA-1^{hi} MHCII^{lo}), mTEC^{hi} (UEA-1^{hi} MHCII^{hi}), endothelial cells (PDGF-Rα⁺) and fibroblasts (CD31⁺) are shown. N, Young female C57Bl/6 mice were pre-treated 5 days before SL-TBI with vehicle (black) or LHRH-Ant (grey) and their thymi were analyzed 7 after irradiation. 0, Absolute numbers of central memory (CM, CD62L^{hi} Cd44^{hi}) and effector memory (EM, CD62L^{lo} Cd44^{hi}) CD4⁺ and CD8⁺ T cells 7, 28 and 42 days after SL-TBI. P, CD5⁺ enriched splenocytes obtained from vehicle and LHRH-Ant treated mice 42 days after irradiation were culture in vitro for cytokine production analysis. Q, Different thymocyte subsets represented in FIG. 4 I. R, The graph shows shortened median survival time in GVHD mice treated with vehicle or LHRH-Ant. Results are expressed as the combined mean±SEM of 8-10 mice for each group representing at least two independent experiments. */̂ (p≦0.05); **/̂̂ (p≦0.01), **̂̂ (p≦0.001), compared with untreated (*) mice and vehicle treated mice (̂).

FIG. 5 shows SSI administration after SL-TBI enhanced thymic regeneration and peripheral reconstitution. 8- to 10-weeks old male C57Bl/6 mice were sub-lethal irradiated and treated 24 hours later with vehicle (black) or Degarelix (grey). Thymi and spleens were analyzed at different time points. A, Absolute number of thymic cellularity and developing thymocytes 7 and 42 days after irradiation. B, Absolute number of thymic stromal cells. C, Total splenocytes and B cells were analyzed at 7, 24 and 48 days after SL-TBI. D, Absolute number of CD4⁺ and CD4⁺N T cells. E, Absolute number of CD8⁺ and CD8⁺N T cells. Results are expressed as the combined mean±SEM of 8-12 mice for each group representing at least two independent experiments. */̂ (p≦0.05); **/̂̂ (p≦0.01), **̂̂ (p≦0.001), compared with untreated (white) (*) mice and vehicle treated mice (̂).

FIG. 6 shows 7 weeks old male and female C57BL/6 mice given a lethal dose of irradiation (845 cGy) and treated 24 hours later with a single dose of vehicle (mannitol, black circle) or Degarelix (grey square). A, Mouse survival was monitored daily in male (left) and female (right) mice. Data were analyzed using the Mantel-Cox log-rank test comparing Degarelix to vehicle alone. ****p≦0.0001, n=40. B, Schematic of experiment protocol for comprehensive analysis of hematopoietic reconstitution after lethal radiation injury. C, Total BM cellularity at days 7, 10, 14 and 24 after lethal TBI. D, complete blood counts (CBC) of peripheral blood at days 7, 10, 14 and 24 after lethal TBI in mice treated with sex steroid inhibition (Degarelix) or vehicle alone (mannitol). At day 24, mannitol group (n=2), all other treatments and time points (n=5).

FIG. 7 shows percent survival of C57BL/6 mice given a lethal dose of irradiation (845 cGy) and treated 48 hours later with a single dose of vehicle (mannitol, black circle) or Degarelix (grey square).

FIG. 8 shows hematopoietic stem and progenitor cell populations (LSK, Sca1⁺ckit⁺) and long-term hematopoietic stem cells (LT-HSC, CD150⁺CD48⁻) in C57BL/6 mice given a lethal dose of irradiation (845 cGy) and treated 24 hours later with a single dose of vehicle (LTBI, black) or Degarelix (LTBI+LHRH-Ant, grey). A, bar graphs (left) show total number of cells for LSK and LT-HSC populations among treatment groups; contour plots (right) show the relative frequency of each LSK and LT-HSC populations among treatment groups. B, bar graphs (top row) show the percentage of LT-HSCs (CD150⁺CD48⁻) for the indicated gated populations (G0, G1, S/G2/M); contour plots (bottom) show the relative frequency of the LT-HSC populations for each gate among both treatment groups.

FIG. 9 shows the repopulating potential of hematopoietic stems cells after a lethal dose of TBI and treatment with Degarelix. Briefly, cells were isolated from the BM of CD45.2⁺ mice 14 days after a lethal dose of TBI (840 cGy) and treatment with a single dose of Degarelix or mannitol (given 24 hours after TBI). Isolated cells were transplanted into otherwise untreated CD45.1⁺ recipients given a lethal dose of TBI (2×550 cGy) to ensure engraftment, along with a small dose (2.5×10⁵) of CD45.1⁺ BM cells to ensure survival of recipients. 28 days after transfer, donor CD45.2⁺ chimerism was analyzed in the blood to examine hematopoietic reconstitution. Top left is a schematic of experiment protocol for comprehensive analysis of repopulating potential in secondary transplant; top right is a bar graph indicating the percentage of CD45.2+ cells among CD45⁺, B220⁺, CD3+ and GR1/Mac1⁺ cell populations from both treatment groups; bottom shows contour plots indicating the relative frequency of CD45.1⁺ and CD45.2⁺ cells from both treatment groups.

FIG. 10 shows the increased expansion of hematopoietic stem cells in C57BL/6 mice after a lethal dose of irradiation (840 cGy). Top left is a schematic of experiment protocol for comprehensive analysis of hematopoietic stem cell expansion using Luciferase-expressing Sca1⁺ckit⁺ hematopoietic stem cells. Bottom left is a bar graph indicating the total number of photons measured in both treatment groups. Right is fluorescent imaging of mice given a lethal dose of irradiation (840 cGy), treated 24 hours later with a single dose of vehicle (LTBI Vehicle, black) or Degarelix (LTBI LHRH-Ant, grey) and implanted 48 hours post treatment with Luciferase-positive hematopoietic stem and progenitor cell populations (Luc⁺LSK [Sca1⁺ckit⁺]).

FIG. 11 shows percent survival in mice given a lethal dose of total body irradiation (TBI) treated with vehicle (mannitol, black circle) or Lupron (grey square).

FIG. 12 shows C57BL/6 mice given a lethal dose of total body irradiation (TBI) one day after surgical castration and treated with a single dose of Degarelix (LHRH-Ant). A, percent survival in mice given total body irradiated (TBI, black circle), mice given total body irradiated plus castration (TBI+castration, grey triangle) and mice given total body irradiated plus castration and treated with Degarelix (TBI+castration+LHRH-Ant, grey inverted triangle). B, ng/mL levels of testosterone, luteinizing hormone (LH) and follicle-stimulating hormone (FSH) at 2, 7 and/or 14 days after a lethal dose of total body irradiation among control (open bars), lethal total body irradiated (LTBI, black bars) and lethal total body irradiation treated with Degarelix (LTBI+LHRH-Ant, grey bars). C, percent survival in mice given total body irradiation (TBI) and treated with Degarelix plus PBS (grey squares), and in mice given total body irradiation (TBI) and treated with Degarelix and a LH analogue (human chorionic gonadotropin, hCG; black circles). Top right is a schematic of experiment protocol for comprehensive analysis of survival of mice given total body irradiation and subsequent combination treatment with a LHRH antagonist and an LH analogue (hCG).

### DEFINITIONS

This invention is not limited to particular methods, and experimental conditions described, as such methods and conditions may vary. It is also to be understood that the terminology used herein is for the purpose of describing particular embodiments only, and is not intended to be limiting, since the scope of the present invention is defined by the claims.

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described. All publications mentioned are hereby incorporated by reference.

The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, siRNAs, shRNAs, DNA/RNA hybrids, antisense oligonucleotides, ribozymes, peptides, peptide mimetics, small molecules, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

As used herein, the term “agonist” refers to any entity that has a positive impact on a function of a protein or hormone of interest. In some embodiments, an agonist directly or indirectly enhances, strengthens, activates and/or increases an activity of a protein of interest. In particular embodiments, an agonist directly interacts with the protein of interest. Such agonists can be, e.g., proteins, chemical compounds, small molecules, nucleic acids, antibodies, drugs, ligands, or other agents. In some embodiments, treatment with an agonist elicits a biological feedback mechanism that results in inhibition of a particular biological response. (e.g., treatment with a hormone receptor agonist that downregulates its cognate receptor and/or results in lower circulating levels of an endogenous hormone). It will be understood by those skilled in the art that the “positive impact” exerted by an agonist need not occur immediately, but is observed within and/or over a relevant period of time.

As used herein, the term “amelioration” refers to the prevention, reduction or palliation of a state, or improvement of the state of a subject. Amelioration includes, but does not require complete recovery or complete prevention of a disease, disorder or condition (e.g., radiation injury). The term “prevention” refers to a delay of onset of a disease, disorder or condition. Prevention may be considered complete when onset of a disease, disorder or condition has been delayed for a predefined period of time.

As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

As used herein, the term “antagonist” refers to an agent that i) inhibits, decreases or reduces the effects of another agent, for example that inactivates a receptor; and/or ii) inhibits, decreases, reduces, or delays one or more biological events, for example, activation of one or more receptors or stimulation of one or more biological pathways. In particular embodiments, an antagonist inhibits activation and/or activity of one or more receptor tyrosine kinases. Antagonists may be or include agents of any chemical class including, for example, small molecules, polypeptides, nucleic acids, carbohydrates, lipids, metals, and/or any other entity that shows the relevant inhibitory activity. An antagonist may be direct (in which case it exerts its influence directly upon the receptor) or indirect (in which case it exerts its influence by other than binding to the receptor; e.g., altering expression or translation of the receptor; altering signal transduction pathways that are directly activated by the receptor, altering expression, translation or activity of an agonist of the receptor). It will be understood by those skilled in the art that the “inhibition, decrease, or reduction” exerted by an antagonist need not occur immediately, but is observed within and/or over a relevant period of time.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the phrase “biologically active” refers to a characteristic of any agent that has activity in a biological system, and particularly in an organism. For instance, an agent that, when administered to an organism, has a biological effect on that organism is considered to be biologically active. In particular embodiments, where a peptide is biologically active, a portion of that peptide that shares at least one biological activity of the peptide is typically referred to as a “biologically active” portion. In certain embodiments, a peptide has no intrinsic biological activity but that inhibits the effects of one or more naturally occurring angiotensin compounds is considered to be biologically active.

As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

As used herein, the term “combination therapy” refers to those situations in which two or more different pharmaceutical agents for the treatment of disease are administered in overlapping regimens so that the subject is simultaneously exposed to at least two agents. In some embodiments, the different agents are administered simultaneously. In some embodiments, the administration of one agent overlaps the administration of at least one other agent. In some embodiments, the different agents are administered sequentially such that the agents have simultaneous biologically activity with in a subject.

As used herein, the term “comparable” refers to two or more agents, entities, situations, sets of conditions, etc. that may not be identical to one another but that are sufficiently similar to permit comparison there between so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

As used herein, the term “complete blood count (CBC),” also known as full blood count (FBC), full blood exam, or blood panel, is a test panel typically ordered by medical professionals that provides information about the cell types and numbers in a patient's blood. In some embodiments, a “complete blood count measure” includes measurement of total white cells, total red cells, hemoglobin, hematocrit, mean corpuscular volume, mean corpuscular hemoglobin, mean corpuscular hemoglobin concentration, red blood cell distribution width, neutrophil granulocytes, lymphocytes, monocytes, eisonophil granulocytes, basophil granulocytes, platelet numbers, and/or mean platelet volume.

As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

As used herein, the term “dosing regimen” (or “therapeutic regimen”), is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, the therapeutic agent is administered continuously over a predetermined period. In some embodiments, the therapeutic agent is administered once a day (QD) or twice a day (BID).

As used herein, the term “functional equivalent” or “functional derivative” denotes a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties, which are similar to that of the substituted amino acid. Desirable similar chemico-physical properties include, similarities in charge, bulkiness, hydrophobicity, hydrophilicity, and the like.

As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of disease or injury as the individual being treated.

As used herein, the term, “inhibitory agent” refers an entity that blocks or reduces the level or activity of a desired target. In some embodiments, an inhibitory agent is characterized in that level or activity of a target is reduced in the presence of the agent as compared with the absence and/or relative to a relevant reference level or activity. A sex steroid inhibitory agent is one that inhibits levels or activity within androgen and/or estrogen signaling systems. In some embodiments, one or more sex steroid inhibitory (SSI) agents may be used to cause a functional sex steroid ablation (SSA). It will be understood by those skilled in the art that an agent may be deemed and/or utilized as an “inhibitory agent” in accordance with the present disclosure even if its inhibitory effects do not occur and/or are not observed immediately; in some embodiments, such effects occur and/or are observed within and/or over a relevant period of time.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

As used herein, the phrase “non-human animal” refers to any vertebrate organism that is not a human. In some embodiments, a non-human animal is a cyclostome, a bony fish, a cartilaginous fish (e.g., a shark or a ray), an amphibian, a reptile, a mammal, and a bird. In some embodiments, a non-human mammal is a primate, a goat, a sheep, a pig, a dog, a cow, or a rodent. In some embodiments, a non-human animal is a rodent such as a rat or a mouse.

The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous, intracerebroventricular, or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

As used herein, the term “prevent” or “prevention”, when used in connection with the occurrence of a disease, disorder, and/or condition, refers to reducing the risk of developing the disease, disorder and/or condition. See the definition of “risk.”

As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

As used herein, the term “protein” refers to a polypeptide (i.e., a string of at least 3-5 amino acids linked to one another by peptide bonds). Proteins may include moieties other than amino acids (e.g., may be glycoproteins, proteoglycans, etc.) and/or may be otherwise processed or modified. In some embodiments “protein” can be a complete polypeptide as produced by and/or active in a cell (with or without a signal sequence); in some embodiments, a “protein” is or comprises a characteristic portion such as a polypeptide as produced by and/or active in a cell. In some embodiments, a protein includes more than one polypeptide chain. For example, polypeptide chains may be linked by one or more disulfide bonds or associated by other means. In some embodiments, proteins or polypeptides as described herein may contain L-amino acids, D-amino acids, or both, and/or may contain any of a variety of amino acid modifications or analogs known in the art. Useful modifications include, e.g., terminal acetylation, amidation, methylation, etc. In some embodiments, proteins or polypeptides may comprise natural amino acids, non-natural amino acids, synthetic amino acids, and/or combinations thereof. In some embodiments, proteins are or comprise antibodies, antibody polypeptides, antibody fragments, biologically active portions thereof, and/or characteristic portions thereof.

As used herein, the term “radiation injury” refers to one or more deleterious health effects caused by exposure to a source of radiation. In some embodiments, a deleterious health effect includes a reduction in the number and/or function of blood cells. In some embodiments, a deleterious health effect includes diminished number or function of hematopoietic stem cells.

The term “reference” is used herein to describe a standard or control agent, individual, population, sample, sequence or value against which an agent, individual, population, sample, sequence or value of interest is compared. In some embodiments, a reference agent, individual, population, sample, sequence or value is tested and/or determined substantially simultaneously with the testing or determination of the agent, individual, population, sample, sequence or value of interest. In some embodiments, a reference agent, individual, population, sample, sequence or value is a historical reference, optionally embodied in a tangible medium. Typically, as would be understood by those skilled in the art, a reference agent, individual, population, sample, sequence or value is determined or characterized under conditions comparable to those utilized to determine or characterize the agent, individual, population, sample, sequence or value of interest

As will be understood from context, a “risk” of a disease, disorder, and/or condition comprises likelihood that a particular individual will develop a disease, disorder, and/or condition (e.g., a radiation injury). In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event (e.g., a radiation injury). In some embodiments a reference sample or group of reference samples are from individuals comparable to a particular individual. In some embodiments, relative risk is 0, 1, 2, 3, 4, 5, 6, 7, 8, 9, 10, or more.

As used herein, the terms “sex hormone,” “sex steroid,” and “sex steroid hormone” are used interchangeably and refer to steroid hormones that interact directly or indirectly with androgen or estrogen receptors, signaling, or function.

In general, a “small molecule” is a molecule that is less than about 5 kilo Daltons (kD) in size. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. In some embodiments, in accordance with the present invention, small molecules are not proteins, polypeptides, oligopeptides, peptides, polynucleotides, oligonucleotides, polysaccharides, glycoproteins, proteoglycans, etc.

The term “specific”, when used herein with reference to an agent or entity having an activity, is understood by those skilled in the art to mean that the agent or entity discriminates between potential targets or states. For example, an agent is said to bind “specifically” to its target if it binds preferentially with that target in the presence of competing alternative targets. In some embodiments, the agent or entity does not detectably bind to the competing alternative target under conditions of binding to its target. In some embodiments, the agent or entity binds with higher on-rate, lower off-rate, increased affinity, decreased dissociation, and/or increased stability to its target as compared with the competing alternative target(s).

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, an individual who is “suffering from” radiation injury has been diagnosed with or displays one or more symptoms of radiation injury.

An individual who is “susceptible to” is at risk for developing the disease, disorder, or condition (e.g. radiation injury). In some embodiments, such an individual is known to have one or more susceptibility factors that are statistically correlated with increased risk of development of the relevant disease, disorder, and/or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition (e.g. irradiation). In some embodiments, a risk of developing a disease, disorder, and/or condition is a population-based risk.

According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom. It is not intended that the present invention be limited only to cases where the symptoms are eliminated. The present invention specifically contemplates treatment such that one or more symptoms is/are reduced (and the condition of the subject is thereby “improved”), albeit not completely eliminated.

As used herein, the term “therapeutically effective amount” refers to an amount of an agent, which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition associated with radiation injury. In some embodiments, treatment may be administered to a subject who does not exhibit signs of radiation injury and/or exhibits only early signs for the purpose of decreasing the risk of developing pathology associated with radiation injury.

### DETAILED DESCRIPTION OF CERTAIN EMBODIMENTS

The present invention demonstrates that administration of an SSI agent can protect against and/or improve recovery from radiation injury.

In some embodiments, an agent that reduces the activity of a sex hormone may be or comprise an agent that reduces the activity of one or more upstream regulators of sex hormone level or activity. In some embodiments, an agent that reduces the activity of a sex hormone may be or comprise an agent that reduces the activity of one or more downstream regulators of sex hormone level or activity. In some embodiments, a useful agent reduces the activity of LH and/or FSH. In some embodiments, a useful agent is or comprises LH or FSH.

The present invention provides, among other things, methods and compositions for post radiation recovery, and particularly for post-radiation hematopoietic recovery.

Among other things, the present disclosure specifically demonstrates that SSI agents administered subsequent to radiation injury can promote hematopoietic recovery and/or survival. For example, the inventors demonstrate that animals treated with an SSI agent after exposure to a sub-lethal dose of radiation show improved recovery characteristics, including for example, in recovery of immune function. To give one specific example, the present disclosure demonstrates more rapid and/or more robust recovery of lymphoid activity after treatment with an SSI agent than under otherwise comparable conditions lacking such treatment.

The present inventors also demonstrate that animals treated with an SSI agent after exposure to an otherwise lethal dose of radiation are able to survive. Furthermore, animals treated with an SSI agent after an otherwise lethal dose of radiation show enhanced hematopoietic recovery as compared with comparable animals not so treated. For example, the present invention demonstrates improved recovery characteristics such as increased number of red blood cells, hemoglobin levels, and hematocrit.

Without wishing to be bound by any particular theory, we note that data provided herein demonstrates that, in some embodiments, (e.g., where an upstream regulator of sex hormone level is utilized), detectable impact on activity (e.g., level) of one or more sex hormones may not be required. For example, the present disclosure demonstrates, among other things, that certain upstream regulators of sex hormone level or activity (e.g., Degarelix, Lupron, etc.), promote hematopoietic recovery even in the absence of sex hormones (e.g., due to castration). Thus, in at least some embodiments, the present disclosure embraces administration of relevant agents independent of detectable effects on sex hormones, including in the absence of sex hormones.

Radiation Injury

Radiation injury is cellular or tissue changes or damage caused by exposure to radiation. Particularly damaging is ionizing radiation from sources such as radioactive chemical compounds, X-rays, nuclear reactors, particle accelerators, nuclear weapons, and the like, which can result in fatality. Exposure to ionizing radiation results in multiple organ dysfunction syndromes that mostly impacts high proliferative cells, such us the cells of the hematopoietic system. In fact, hematopoietic cells are highly sensitive to radiation damage and relatively low levels of exposure can result in bone marrow failure and potentially lethal anemia, hemorrhage or infections. Typical hematopoietic pathology resulting from radiation injury includes a rapid reduction in the cell count of lymphocytes, granulocytes, thrombocytes, and reticulocytes and in the committed progenitors of these lineages, ultimately leading to neutropenia, thrombocytopenia, anemia and death. The body's supply of blood cells is replenished by hematopoietic stem cells that in the adults mostly reside in the bone marrow. The bone marrow is the most classically recognized target of acute radiation exposure that in humans appears at even very low radiation doses such as 1 Gy.

As a result, there is a need for treatments that can rapidly restore hematopoietic stem cell activity and promote recovery from radiation injury. In particular, there is a need for therapeutic treatments that are effective when administered subsequent to radiation exposure.

Treatment of Radiation Injury

Despite the large body of intensive research to identify effective treatment for the mitigation of radiation injury, currently available approaches are still limited and only G-CSF is currently stockpiled for this purpose. Several other cytokines and growth factors are under investigation for this purpose, such as IL-1, IL-3, IL-7, IL-11, IL-12, TNF-alpha, SCF, EGF, KGF, C-CSF and GM-CSF. Some have shown radioprotective properties when administered before radiation exposure. Although their presence in the body may protect from radiation effects, mainly by boosting recovery, few of them have shown beneficial effects when administered after radiation exposure, precluding their use in a radiation accident scenario. In fact, one of the major challenges in this situation is the unpredictable nature of the event resulting in a need for medical countermeasures and treatments that are not immediately available. Therefore the identification of medical countermeasures active when administered at least 24 h after radiation exposure represents a major unmet challenge. This is particularly critical for radiation accidents affecting a very large number of victims, such as during the worst-case scenario of a nuclear weapon detonation, a nuclear accident or a terrorist attack.

The colony stimulating factors G-CSF and GM-CSF, FDA approved for the treatment of neutropenia, have shown positive effects in neutrophil recovery and overall survival in different animal models when administered after irradiation. However, the use of CSFs comes with important drawbacks: CSFs are expensive and current therapeutic protocols are based on daily administration. In addition, CSF treatment can result in important side effects, such as antigenicity, bone pain, splenomegaly and treatment may exacerbate pre-exciting inflammatory conditions. Furthermore, although CSFs provide significant benefit in neutrophil recovery, this treatment is not effective in protecting and enhancing the recovery of broader hematopoiesis as lymphoid, thromboid and erythroid lineages, which are critical for effective recovery following radiation injury, are unaffected by G-CSF.

Although IL-7 and KGF have both been used to enhance immune recovery in recipients of hematopoietic stem cell transplantation when given prior to exposure to cytoreductive chemotherapy or radiation therapy, there is no evidence that they will work for promoting recovery when given after radiation injury. Moreover, given that the targets of these cytokines are lymphoid precursors (in the case of IL-7) and epithelial cells (in the case of KGF) there is no basis to expect that they would have a positive impact on hematopoietic stem cells and their protection or recovery following radiation injury.

An ideal treatment for mitigation of radiation causalities should be 1) active when administered at least 24 h after irradiation, 2) able to enhance lymphoid and erythromyeloid recovery, and 3) readily available.

Sex Steroid Inhibitory Agents

In some embodiments, an SSI is administered to inhibit levels or activity within androgen and/or estrogen signaling systems. In some embodiments, one or more sex steroid inhibitory (SSI) agents may be used to cause a functional sex steroid ablation (SSA).

In some embodiments, SSI agents that may be utilized in accordance with the present invention include small molecules, antibodies, antibody fragments, aptamers, siRNAs, shRNAs, DNA/RNA hybrids, antisense oligonucleotides, ribozymes, peptides, peptide mimetics, lipids, small molecules, etc.

In some embodiments, an SSI agent is an LHRH agonist. Exemplary LHRH agonists, include, but are not limited to, goserelin, leuprolide, triptorelin, buserelin, nafarelin, deslorelin, histrelin, and the like.

In some embodiments, an SSI agent is an LHRH antagonist. Exemplary LHRH antagonists include, but are not limited to, cetrorelix, ganirelix, abarelix, degarelix, and the like.

In some embodiments, an SSI agent is an androgen receptor antagonist. In some embodiments, an androgen receptor antagonist is non-steroidal. In some embodiments, an androgen receptor antagonist is steroid. Exemplary androgen receptor antagonists include, but are not limited to, enzalutamide (MDV3100), cyproterone acetate, spironalactone, dropirenone, flutamide, bicalutamide, nilutamide, PF 998425, and the like.

In some embodiments, an SSI agent is an estrogen receptor antagonist. In some embodiments, an estrogen receptor antagonist is a selective estrogen receptor modulator (SERM). In some embodiments, an SSI agent is a substantially pure estrogen receptor antagonist. Exemplary estrogen receptor antagonists include, but are not limited to, fulvestrant, tamoxifen, clomifine, raloxifene, ormeloxifene, tamoxifen, toremifene, lasofoxifene, ospemifene, afimoxifene, arzoxifene, bazedoxifene, and the like.

In some embodiments, an SSI agent is an upstream or downstream regulator of a sex steroid or sex steroid activity. In some embodiments, an SSI agent is or comprises leutinizing hormone (LH), follicle-stimulating hormone (FSH) or leutinizing hormone releasing hormone (LHRH), or an analog, a regulator, or a modulator thereof. Exemplary upstream or downstream regulator of a sex steroid include those affect one or more activities of a sex steroid (e.g., expression, modulation of a sex steroid target, modulation, etc.).

In some embodiments, the invention provides for the identification and/or characterization of novel SSI agents having an ability to promote recovery from radiation injury in an in vitro or in vivo assay as exemplified herein. In some embodiments, novel or newly identified SSI agents have an ability to promote hematopoietic recovery. In some embodiments, an SSI agent is identified or characterized as having an ability to increase hematopoietic stem cell number or activity when administered to a subject exposed to irradiation in comparison to hematopoietic stem cell number or activity in a comparable subject exposed to irradiation and not administered an SSI agent. In some embodiments, an SSI agent is identified or characterized as having an ability to promote survival of a subject exposed to irradiation as compared to a subject exposed to irradiation and not administered an SSI agent. In some embodiments, an SSI agent is identified or characterized as having an ability to increase blood count measurements in a subject exposed to irradiation as compared to a subject exposed to irradiation and not administered an SSI agent.

In some embodiments, the present invention provides for in vitro or in vivo screening methods that identify and/or characterize SSI agents. In some embodiments, agents identified and/or characterizes according to such methods are, comprise, or affect sex steroid; in some embodiments they are or comprise one or more upstream regulators of sex steroid (hormone). In some certain embodiments, assays detect or utilize direct interaction with a sex steroid and/or with an upstream regulator thereof (e.g., by binding, such as by an antibody); in some embodiments, assays detect or utilize indirect activity (e.g., as with siRNA or other agents that modulate expression of a sex steroid and/or of an upstream regulator thereof).

Those skilled in the art, reading the present disclosure, will appreciate that any of a variety of available assay formats may be utilized. For example, screening methods may utilize cell cell-free, cell-based, tissue based, organ-based, and/or animal assays. In some embodiments, assays (particularly in vitro assays) may be performed in the solid state; in some embodiments they are performed in the liquid state. Any of a variety of available readouts and/or detection systems may be employed.

Administration of SSI Agents

In the methods of the invention, an SSI agent is typically administered to the individual alone, or in compositions or medicaments comprising the SSI (e.g., in the manufacture of a medicament for the treatment of the disease), as described herein. The compositions can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interference with their activity. In some embodiments, a water-soluble carrier suitable for intravenous administration is used.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The composition or medicament can be formulated in accordance with the routine procedures as a pharmaceutical composition adapted for administration to human beings. For example, in some embodiments, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

SSI agents can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

SSI agents (or a composition or medicament containing one or more SSI agents is administered by any appropriate route. In some embodiments, SSI agents are administered intravenously. In some embodiments, SSI agents are administered subcutaneously. In some embodiments, SSI agents are administered by direct administration to a target tissue, such as heart or muscle (e.g., intramuscular), or nervous system (e.g., direct injection into the brain; intraventricularly; intrathecally). Alternatively, SSI agents (or a composition or medicament containing SSI agents can be administered parenterally, transdermally, or transmucosally (e.g., orally or nasally). More than one route can be used concurrently, if desired.

SSI agents (or a composition or medicament containing SSI agents, can be administered alone, or in conjunction with other SSI agents. The term, “in conjunction with,” indicates that a first SSI agent is administered prior to, at about the same time as, or following another SSI agent. For example, a first SSI agent can be mixed into a composition containing one or more different SSI agents, and thereby administered contemporaneously; alternatively, the agent can be administered contemporaneously, without mixing (e.g., by “piggybacking” delivery of the agent on the intravenous line by which the SSI agent is also administered, or vice versa). In another example, the SSI agent can be administered separately (e.g., not admixed), but within a short time frame (e.g., within 24 hours) of administration of the SSI agent.

SSI agents (or a composition or medicament containing SSI agents are administered in a therapeutically effective amount (i.e., a dosage amount that, when administered at regular intervals, is sufficient to treat the radiation injury, such as by ameliorating symptoms associated with the radiation injury, preventing or delaying the onset of the radiation injury, and/or also lessening the severity or frequency of symptoms of the radiation injury. As used herein, the therapeutic effective amount is also referred to as therapeutic effective dose or therapeutic effective dosage amount. The dose, which will be therapeutically effective for the treatment of radiation injury, will depend on the nature and extent of radiation exposure, and can be determined by standard clinical techniques. In addition, in vitro or in vivo assays may optionally be employed to help identify optimal dosage ranges, such as those exemplified below. The precise dose to be employed will also depend on the route of administration, and the magnitude of the injury, and should be decided according to the judgment of a practitioner and each patient's circumstances. Effective doses may be extrapolated from dose-response curves derived from in vitro or animal model test systems (e.g., as described by the U.S. Department of Health and Human Services, Food and Drug Administration, and Center for Drug Evaluation and Research in “Guidance for Industry: Estimating Maximum Safe Starting Dose in Initial Clinical Trials for Therapeutics in Adult Healthy Volunteers”, Pharmacology and Toxicology, July 2005.

In some embodiments, the therapeutically effective amount of an SSI agent can be, for example, more than about 0.01 mg/kg, more than about 0.05 mg/kg, more than about 0.1 mg/kg, more than about 0.5 mg/kg, more than about 1.0 mg/kg, more than about 1.5 mg/kg, more than about 2.0 mg/kg, more than about 2.5 mg/kg, more than about 5.0 mg/kg, more than about 7.5 mg/kg, more than about 10 mg/kg, more than about 12.5 mg/kg, more than about 15 mg/kg, more than about 17.5 mg/kg, more than about 20 mg/kg, more than about 22.5 mg/kg, or more than about 25 mg/kg body weight. In some embodiments, a therapeutically effective amount can be about 0.01-25 mg/kg, about 0.01-20 mg/kg, about 0.01-15 mg/kg, about 0.01-10 mg/kg, about 0.01-7.5 mg/kg, about 0.01-5 mg/kg, about 0.01-4 mg/kg, about 0.01-3 mg/kg, about 0.01-2 mg/kg, about 0.01-1.5 mg/kg, about 0.01-1.0 mg/kg, about 0.01-0.5 mg/kg, about 0.01-0.1 mg/kg, about 1-20 mg/kg, about 4-20 mg/kg, about 5-15 mg/kg, about 5-10 mg/kg body weight. In some embodiments, a therapeutically effective amount is about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg about 1.4 mg/kg, about 1.5 mg/kg, about 1.6 mg/kg, about 1.7 mg/kg, about 1.8 mg/kg, about 1.9 mg/kg, about 2.0 mg/kg, about 2.5 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, body weight, or more. In some embodiments, the therapeutically effective amount is no greater than about 30 mg/kg, no greater than about 20 mg/kg, no greater than about 15 mg/kg, no greater than about 10 mg/kg, no greater than about 7.5 mg/kg, no greater than about 5 mg/kg, no greater than about 4 mg/kg, no greater than about 3 mg/kg, no greater than about 2 mg/kg, or no greater than about 1 mg/kg body weight or less. In some embodiments, the effective dose for a particular individual is varied (e.g., increased or decreased) over time, depending on the needs of the individual.

In yet another example, a loading dose (e.g., an initial higher dose) of a therapeutic composition may be given at the beginning of a course of treatment, followed by administration of a decreased maintenance dose (e.g., a subsequent lower dose) of the therapeutic composition.

Without wishing to be bound by any theories, it is contemplated that a loading dose clears out the initial and, typically massive, accumulation of fatty materials in tissues (e.g., in the liver), and maintenance dosing prevents buildup of fatty materials after initial clearance.

It will be appreciated that a loading dose and maintenance dose amounts, intervals, and duration of treatment may be determined by any available method, such as those exemplified herein and those known in the art. In some embodiments, a loading dose amount is about 0.01-1 mg/kg, about 0.01-5 mg/kg, about 0.01-10 mg/kg, about 0.1-10 mg/kg, about 0.1-20 mg/kg, about 0.1-25 mg/kg, about 0.1-30 mg/kg, about 0.1-5 mg/kg, about 0.1-2 mg/kg, about 0.1-1 mg/kg, or about 0.1-0.5 mg/kg body weight. In some embodiments, a maintenance dose amount is about 0-10 mg/kg, about 0-5 mg/kg, about 0-2 mg/kg, about 0-1 mg/kg, about 0-0.5 mg/kg, about 0-0.4 mg/kg, about 0-0.3 mg/kg, about 0-0.2 mg/kg, about 0-0.1 mg/kg body weight. In some embodiments, a loading dose is administered to an individual at regular intervals for a given period of time (e.g., 1, 2, 3, 4, 5, 6, 7, 8, 9, 10, 11, 12 or more months) and/or a given number of doses (e.g., 1, 2, 3, 4, 5, 6, 7, 8, 9, 10, 15, 20, 25, 30 or more doses), followed by maintenance dosing. In some embodiments, a maintenance dose ranges from 0-2 mg/kg, about 0-1.5 mg/kg, about 0-1.0 mg/kg, about 0-0.75 mg/kg, about 0-0.5 mg/kg, about 0-0.4 mg/kg, about 0-0.3 mg/kg, about 0-0.2 mg/kg, or about 0-0.1 mg/kg body weight. In some embodiments, a maintenance dose is about 0.01, 0.02, 0.04, 0.06, 0.08, 0.1, 0.2, 0.3, 0.4, 0.5, 0.6, 0.7, 0.8, 0.9, 1.0, 1.2, 1.4, 1.6, 1.8, or 2.0 mg/kg body weight. In some embodiments, maintenance dosing is administered for 1, 2, 3, 4, 5, 6, 7, 8, 9, 10, 11, 12 or more months. In some embodiments, maintenance dosing is administered for 1, 2, 3, 4, 5, 6, 7, 8, 9, 10 or more years. In some embodiments, maintenance dosing is administered indefinitely (e.g., for life time).

A therapeutically effective amount of an SSI agent (or composition or medicament containing an SSI agent or agents may be administered as a one time dose or administered at intervals, depending on the nature and extent of the radiation injury effects, and on an ongoing basis. Administration at an “interval,” as used herein, indicates that the therapeutically effective amount is administered periodically (as distinguished from a one-time dose). The interval can be determined by standard clinical techniques. In some embodiments, an SSI agent is administered bimonthly, monthly, twice monthly, triweekly, biweekly, weekly, twice weekly, thrice weekly, or daily. The administration interval for a single individual need not be a fixed interval, but can be varied over time, depending on the needs and rate of recovery of the individual.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

The invention additionally pertains to a pharmaceutical composition comprising an SSI agent, as described herein, in a container (e.g., a vial, bottle, bag for intravenous administration, syringe, etc.) with a label containing instructions for administration of the composition for treatment of radiation injury.

In some embodiments, an SSI agent is administered to a subject contemporaneously with radiation exposure. In some embodiments, a SSI agent is administered subsequent to radiation exposure. In some embodiments, a SSI agent is administered at least 1, 6, 12, 24, 48, 72, 120, or 168 hours subsequent to radiation exposure.

### EXAMPLES

The following examples are provided so as to describe to those of ordinary skill in the art how to make and use methods and compositions of the invention, and are not intended to limit the scope of what the inventors regard as their invention. Unless indicated otherwise, temperature is indicated in Celsius, and pressure is at or near atmospheric.

### Example 1

### Androgens Regulate Thymopoiesis by Direct Transcriptional Control of Notch Ligands

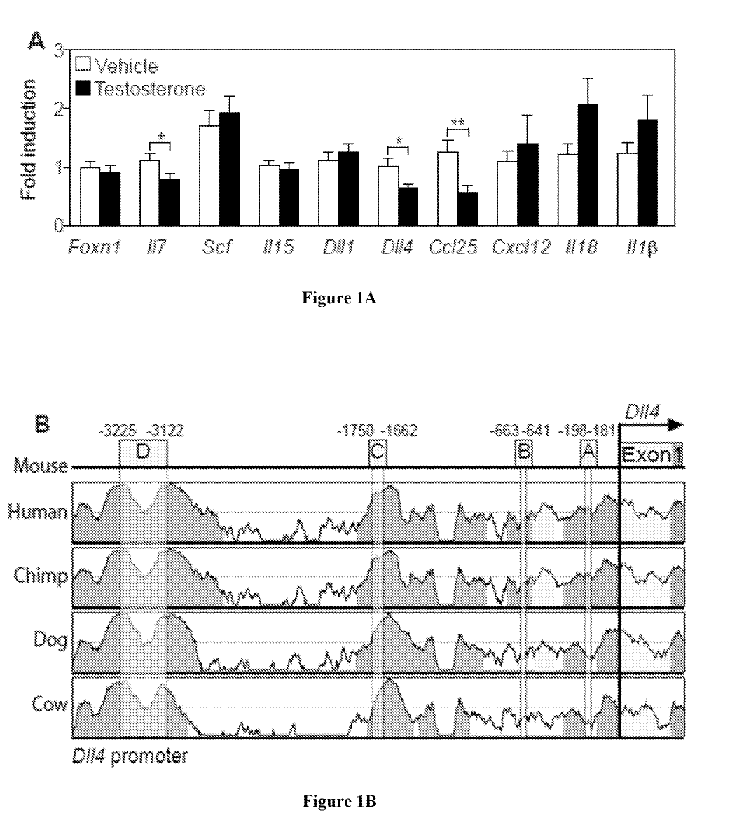

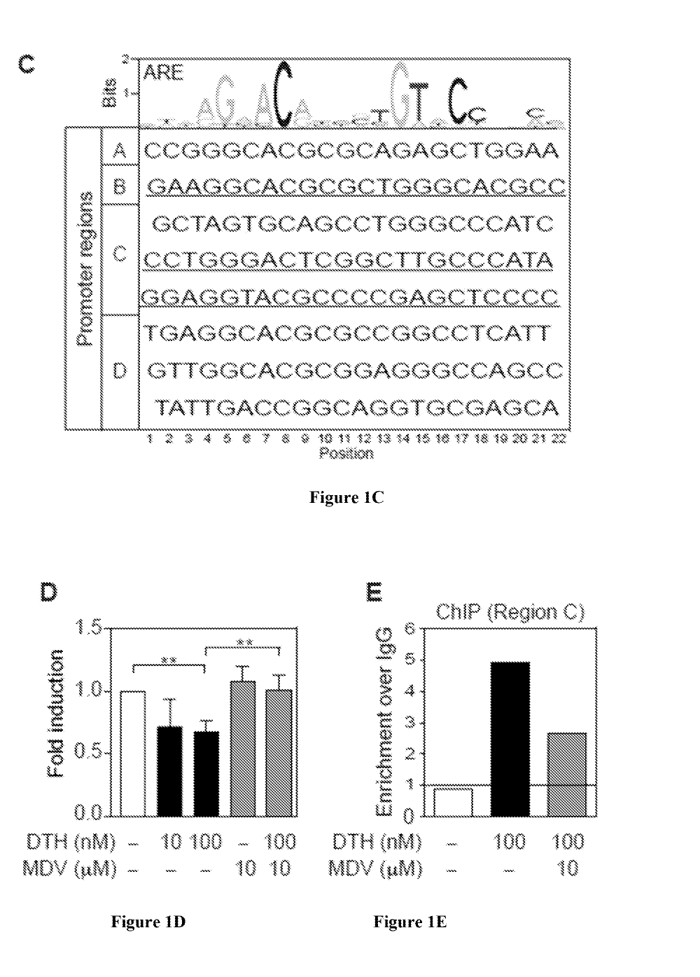

This example illustrates the discovery that androgens directly control transcription of Notch ligands. Previous studies have demonstrated that expression of androgen receptor (AR) in the thymic stromal compartment is indispensable for thymic rebound after surgical castration (Lai et al., 2013; Olsen et al., 2001). Given the primary role of the thymic stroma in thymopoiesis, we investigated the expression levels of key stromal-derived thymopoietic factors after testosterone treatment to identify candidate genes regulated by androgen signaling. Consistent with previous studies (Goldberg et al., 2007; Williams et al., 2008), we found significant down regulation of Il7 and Ccl25 after androgen treatment (FIG. 1A). We also found significantly lower levels of the Notch ligand Dll4.

One mechanism that AR uses to regulate its target genes is through its interaction with specific palindromic DNA binding consensus sequences containing two asymmetrical elements separated by a 3-bp spacer, 5′-GGA/TACANNNTGTTCT-3′ (SEQ ID NO: 1) (Roche et al., 1992). To determine if the observed transcriptional changes were the consequence of direct genomic regulation by the AR, we scrutinized the promoters of Il7, Ccl25 and Dll4 for putative AR elements (AREs). Although we did not detect any AREs in the promoters of Il7 or Ccl25, suggesting an indirect mechanism of regulation, we identified eight AREs that were over-represented in the Dll4 promoter, six of which were equally distributed in two regions (FIGS. 1, B and C). Given that Dll4 is mainly expressed by cortical thymic epithelial cells (cTECs) (Koch et al., 2008), we treated the cTEC cell line C9 with the androgen dihydrotestosterone (DTH) to explore the impact of sex steroids on Dll4 expression. We found that C9 cells treated with DTH exhibited a decrease in the expression of Dll4 24 h after treatment (FIG. 1 D). Sex steroids directly caused this observation, as the reduction in Dll4 expression was abrogated in the presence of the direct AR inhibitor MDV3100. To demonstrate that AR directly regulates Dll4 transcription through physical interaction with its promoter, we performed chromatin-immunoprecipitation (ChIP) using an antibody specific for AR in C9 cells. We dissected the Dll4 promoter in 4 regions according to the putative AREs (FIGS. 1B, 1C) and analyzed binding in each region with specific primers. We found five-fold enrichment of region C immunoprecipitated by AR antibody 2 h after DTH treatment, in which 3 AREs clustered consecutively over a short sequence of 90 bp (FIG. 1E). Importantly, pre-treatment with the AR inhibitor MDV3100 partially impeded this interaction. Taken together, these findings reveal that AR negatively modulates Dll4 expression through physical interaction with its promoter. These findings are consistent with the observation that Dll4 expression decreases with age (Itoi et al., 2007), and suggest that androgen regulation of Dll4 may represent a key process during thymic involution.

### Example 2

### LHRH Receptor Antagonists Promote Thymopoiesis without the Degenerative Phase Observed with LHRH Agonists

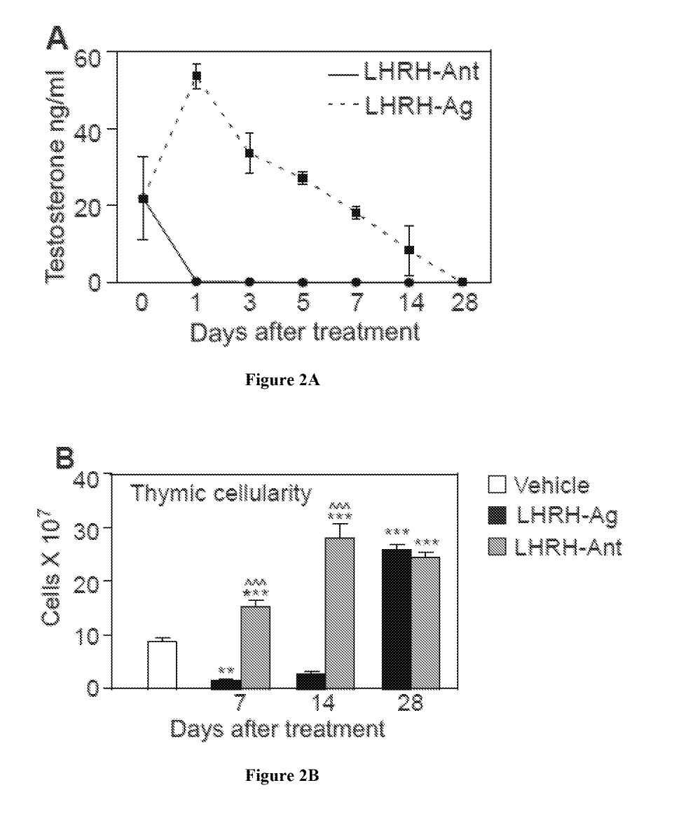

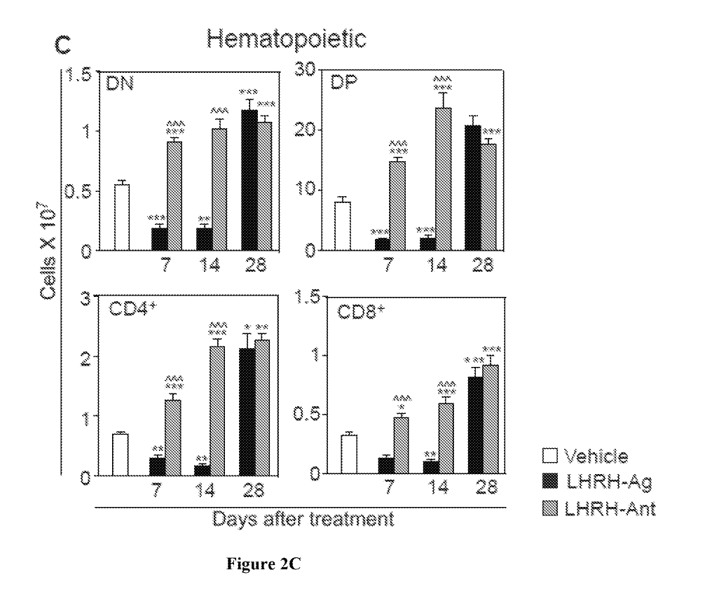

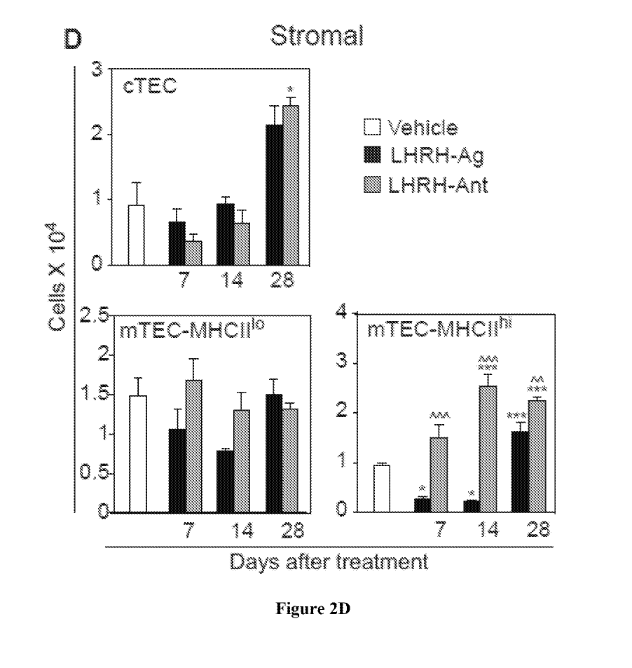

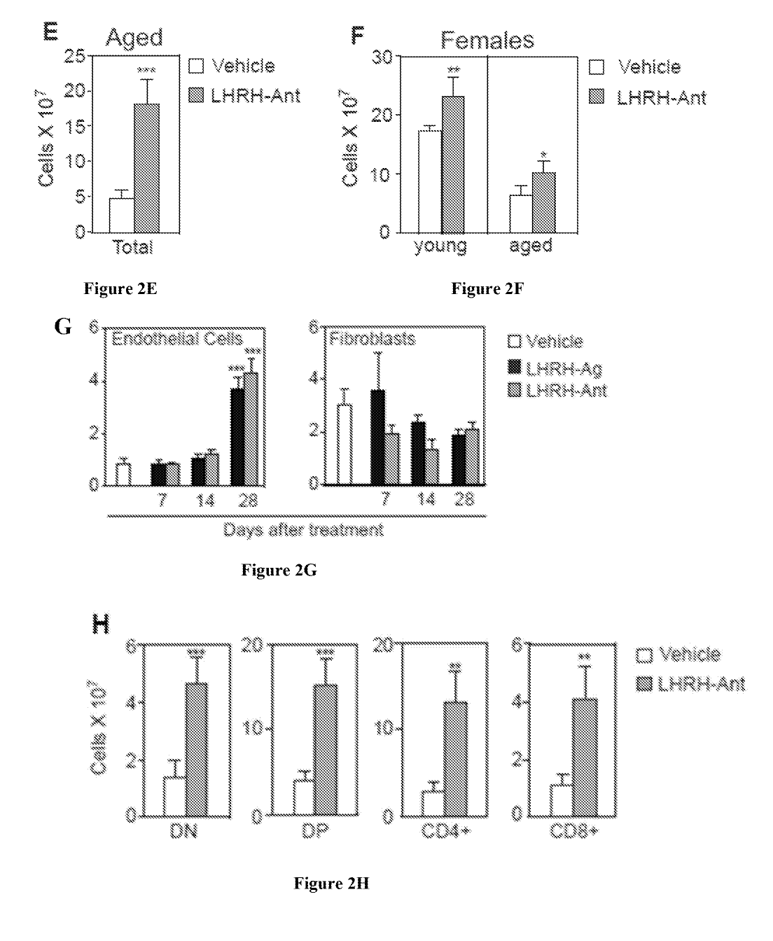

This example illustrates that SSI agents are useful to promote thymopoiesis and that LHRH antagonists may offer certain advantages over LHRH agonists. Clinically, one of the most potent ways of inducing castrate levels of sex steroids is to use an analog for the LHRH receptor (LHRH-R). However, due to its fundamental mechanism of initial sensitization of the LHRH-R, there is an early surge in sex steroids before castrate levels are eventually reached (van Poppel and Nilsson, 2008). In contrast to LHRH agonists (LHRH-Ag), LHRH antagonists (LHRH-Ant) cause immediate cessation of sex steroid production and castrate levels of circulating sex steroids within 24-48 hours (FIG. 2A).

LHRH-Ag treatment caused a dramatic degenerative effect on thymic cellularity at early time points (day 7 and 14) after treatment (FIG. 2B), likely due to the initial increase in testosterone level. Conversely, LHRH-Ant mediated a rapid increase in thymic size compared to untreated control and LHRH-Ag treated mice as early as day 7 after treatment. This rapid increase in thymic size after treatment is consistent with previous studies demonstrating that thymic cellularity of surgically castrated B6 mice, which also exhibit almost immediate cessation of sex steroids, increased within 7 days after surgery (Heng et al., 2005; Sutherland et al., 2005). The effects observed on total thymic cellularity in LHRH-Ant treated mice were reflected by an increase in all developing thymocytes subsets, at days 7 and 14, in contrast to the considerable depletion of these cells following LHRH-Ag treatment (FIG. 2C). By day 28 after treatment, LHRH-Ant and LHRH-Ag demonstrated similar thymic enlargement. Within the stromal compartment, LHRH-Ant caused profound expansion of MHC class II^{high} medullary thymic epithelial cells (mTEC^{hi}), with little impact on MHC class II^{lo} mTECs (mTEC^{lo})(FIG. 2D). In contrast, treatment with LHRH-Ag caused an initial decrease in both mTEC^{lo} and mTEC^{hi} with a more profound effect on mTEC^{hi} at day 7 and 14 after treatment. By 28 days after treatment, the numbers of mTEC^{hi} and mTEC^{lo} in LHRH-Ag treated mice were comparable to LHRH-Ant treated animals. Interestingly, analysis of other thymic stromal subsets, including cTECs, fibroblasts and endothelial cells, did not reveal significant differences between LHRH-Ag and LHRH-Ant treated mice (FIGS. 2D, 2G). Together, these data indicate that LHRH-Ant promote thymic enhancement, without the characteristic LHRH-Ag-induced drop in cellularity.

### Example 3

### LHRH Antagonists Reverse Physiologic Decreases in Thymic Cellularity in Aged Males and Females

This example illustrates that SSI agents are useful to reverse decreases in thymic cellularity. We next investigated the capacity of LHRH-Ant to reverse physiologic decreases in thymic cellularity in the setting of aging. Importantly, 9 month-old male mice, which already have considerable age-related thymic involution (Heng et al., 2005), responded to the regenerative effects of LHRH-Ant with increased levels of total thymic cellularity and all thymic subsets compared to control mice (FIGS. 2E, 2H). LHRH-Ant did not significantly impact on cTECs but showed a robust expansion in the medulla, represented by both mTEC^{hi} and mTEC^{lo} populations (FIG. 21). In addition to the well-known effects of androgens on thymopoiesis, estrogen has also been shown to negatively impact thymic function and can contribute to its involution (Zoller and Kersh, 2006). Given the direct influence of LHRH on both androgens and estrogens, and the profound effect of LHRH antagonism on the regeneration of thymopoiesis in young and aged male mice, we tested the efficacy of LHRH-Ant in female mice, which can also be applied to young and aged females for thymic regeneration. Consistent with our findings in male mice, and valuable for its wider clinical application, we found that LHRH-Ant treatment caused a significant increase in thymic cellularity 28 days after treatment in both young and aged female mice (FIG. 2F).

### Example 4

### Sex Steroid Inhibition Via LHRH-Antagonism Leads to an Increase in the Expression of Dll4 and Downstream Notch Targets

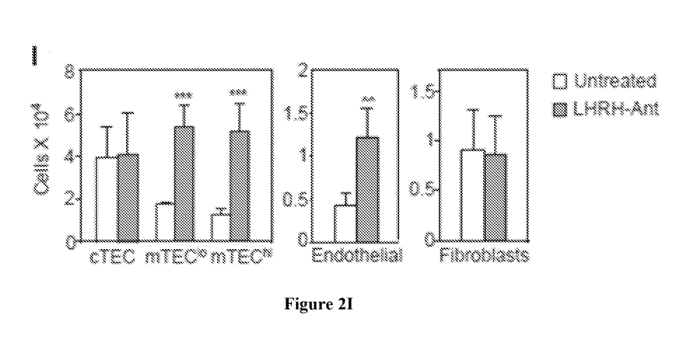

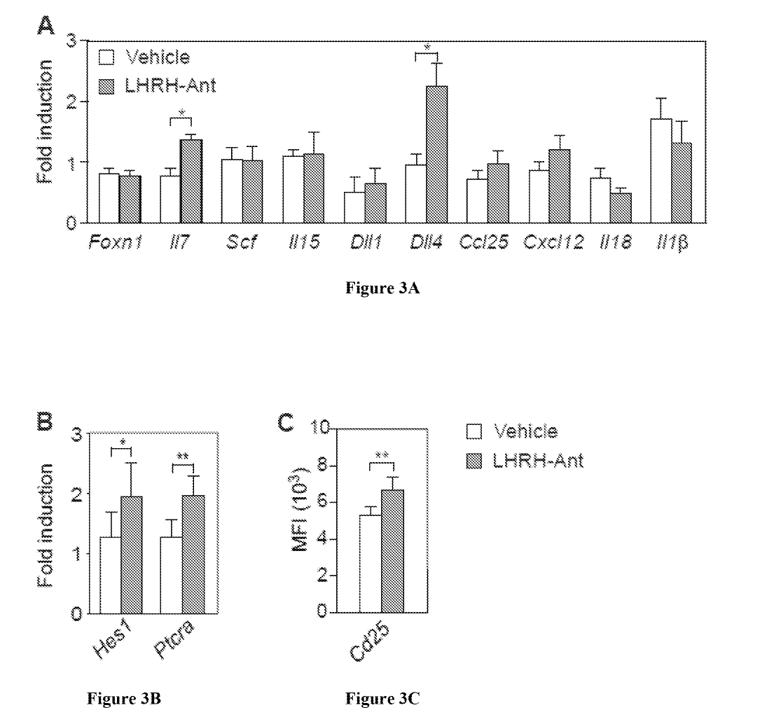

This example illustrates a mechanism by which SSI may affect Notch mediated signaling. Given that SSA impacts the stromal microenvironment, we examined the expression of key thymopoietic factors in thymic stromal cells 7 days after LHRH-Ant treatment. In contrast to previous reports (Williams et al., 2008), we did not see a significant increase in the expression of Ccl25, although this could be due to differences in the experimental approach. Consistent with our data using testosterone (FIG. 1A), we found significant up-regulation in the expression of Il7, which is required for SSA-mediated thymic regeneration (Goldberg et al., 2007) and Dll4, after LHRH-Ant treatment. Consistent with an association between sex steroids and Notch signaling, we found the expression levels of the downstream Notch targets (Hes1, Ptcra and CD25) were also significantly elevated in developing T cells after treatment with LHRH-Ant (FIGS. 3B, 3C).

### Example 5

### LHRH Antagonist Administration Protects Thymic Stroma and Enhances Thymopoiesis after Immune Recovery

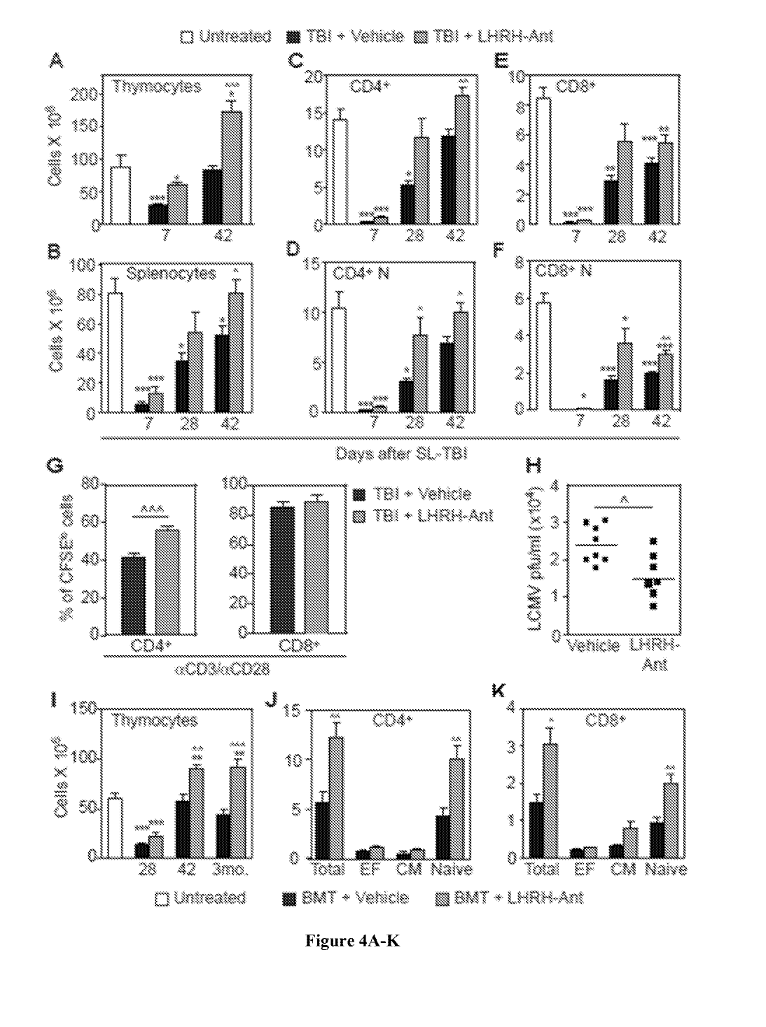

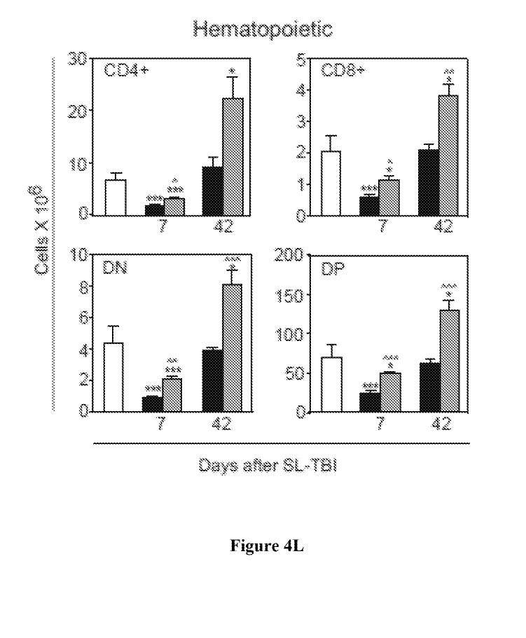

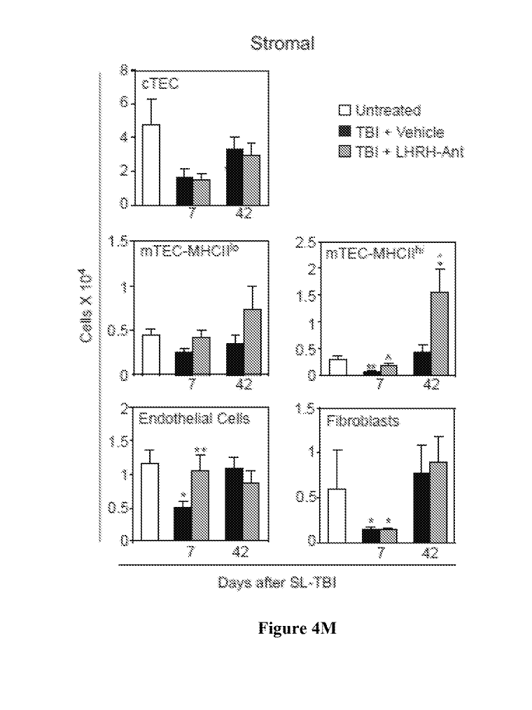

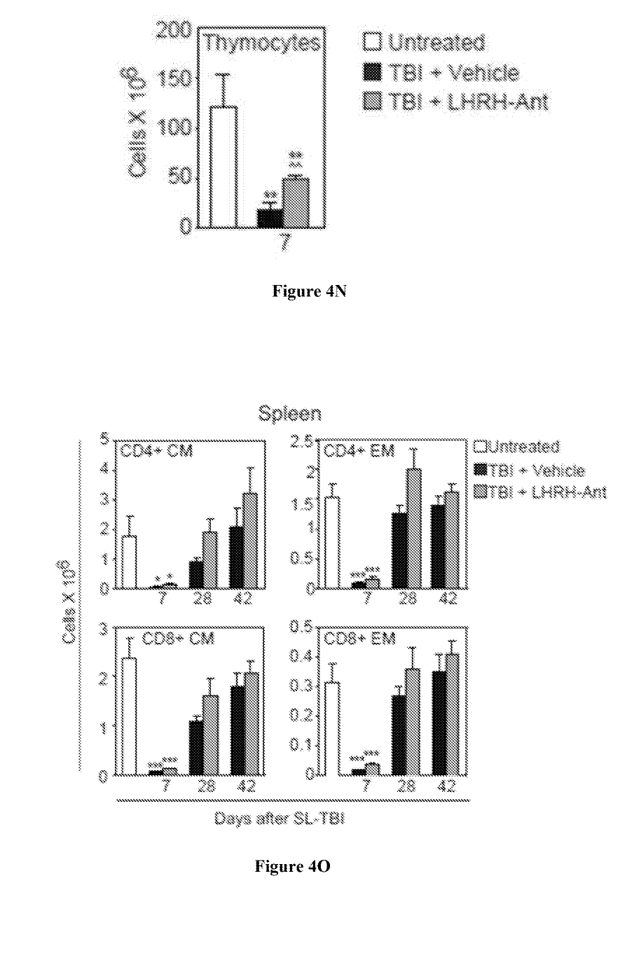

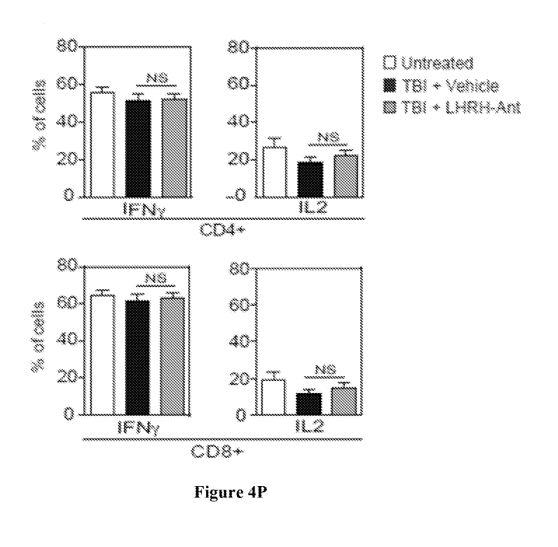

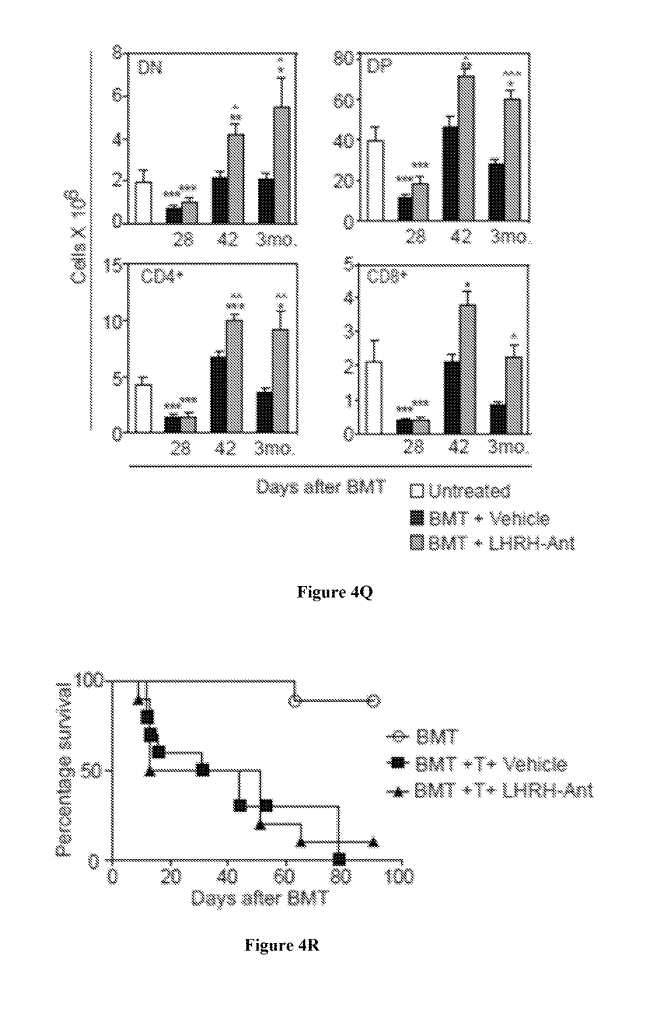

This example illustrates that an SSI agent accelerates immune system recovery from radiation injury. We tested if treatment with LHRH-Ant could accelerate thymic reconstitution and peripheral immune regeneration in mice after immune injury caused by sublethal total body irradiation (SL-TBI). Thymic cellularity was strongly depleted 7 days after SL-TBI and returned to steady-state levels untreated level by day 42 (FIG. 4A). Administration of LHRH-Ant resulted in enhanced recovery of thymic cellularity starting at day 7 after SL-TBI, and remained significantly enlarged at day 42 compared to control mice (FIG. 4A). Analysis revealed increases in all thymocyte subsets (FIG. 4L) and, consistent with our data under steady-state conditions, protection of mainly mTEChi and endothelial cells from radiation injury (FIG. 4M). Importantly, accelerated thymic recovery was also evident 7 days after SL-TBI in female mice treated with LHRH-Ant (FIG. 4N).

We next sought to determine if the enhanced thymopoiesis after LHRH-Ant treatment translated into improved immune recovery in the periphery after SL-TBI. Total splenic cellularity was increased by day 28 after SL-TBI, comprised primarily of increased numbers of both CD4⁺ and CD8⁺ T cells (FIG. 4B), with naïve (CD62L⁺CD44⁻) T cells the most affected by LHRH-Ant treatment (FIGS. 4C-F, 4O). Functionally, although there were no significant differences in the production of IFNγ and IL-2 (FIG. 4P), the proliferation of CD4⁺ T cells upon T cell receptor (TCR) ligation was significantly increased in those derived from LHRH-Ant treated mice (FIG. 4G). This result may be due to the increased number of recent thymic emigrant CD62L⁺CD44⁻ naïve T cells exported from the thymus and to their increased capacity to proliferate following TCR engagement.

One of the major clinical challenges that immunocompromised patients encounter is their increased susceptibility to infection (Wils et al., 2011). To assess the function of T cells and their ability to clear an infection, mice treated with vehicle or LHRH-Ant were challenged with lymphocytic choriomeningitis virus (LCMV) 14 days after SL-TBI. Mice treated with LHRH-Ant showed a significantly lower viral burden compared to vehicle treated mice at day 8 after infection (FIG. 4H), suggesting that the pool of cells being produced by the thymus are functionally superior. These studies are in agreement with a recent report showing that surgical castration can improve T cell functionality and viral clearance in aged mice (Heng et al., 2012).

### Example 6

### LHRH Antagonist Treatment Rapidly Restores Thymopoiesis after Allo-HSCT and Boosts Peripheral Immune Reconstitution

This example illustrates that SSI agents are useful to regenerate the thymus and immune system. We, and others, have previously shown that SSA using LHRH-Ag promotes recovery from autologous and allo-HSCT (Goldberg et al., 2007; Sutherland et al., 2008). We therefore investigated the effects of LHRH-Ant pretreatment on male C57BL/6 allo-HSCT recipients on thymic and peripheral reconstitution. Thymic cellularity was significantly increased in LHRH-Ant treated recipients at day 42, and sustained for at least 3 months after transplant (FIG. 4I). The analysis of developing thymocytes revealed a significant increase in all subsets for at least 3 months after transplant, suggesting that the effects of LHRH-Ant were long-lasting (FIG. 4Q).

Characterization of peripheral T cell reconstitution 3 months after transplant showed a significant increase in the number of CD4⁺ and CD8⁺ T cell subsets (FIG. 4J, 4K). Of note, the most abundant populations among these peripheral T cells subsets were naïve T cells, indicating a robust thymopoiesis in LHRH-Ant treated mice compared to controls. Many strategies for boosting immune reconstitution could also undesirably increase the risks of graft-versus-host disease (GVHD) in allo-HSCT recipients. We therefore evaluated the impact of LHRH-Ant treatment using an established murine GVHD model. We transplanted B10.BR donor (BM) cells with or without T cells into lethally irradiated C57BL/6 recipients and we did not observe significant difference in the GVHD mortality between LHRH-Ant treated and control mice (FIG. 4R). LHRH-Ant treatment therefore enhances thymic output and peripheral T cell function without exacerbating post-transplant complications.

Although it is well known that castration can reverse age-related thymic involution, increase thymic function and boost T cell output in the periphery in mouse and human, the mechanisms underlying these effects are still poorly understood. Our study offers an important novel mechanism by which SSA mediates its effect. We present evidence of a direct negative regulation of Notch signaling by androgens. Clinically, LHRH-Ag represents the most common agent used for androgen deprivation therapy in prostate cancer patients: however, their use is limited by the initial surge in sex steroids they cause. Here, we demonstrate that LHRH-Ant can be used as a therapeutic for regeneration of the thymus and immune system.

### Example 7

### LHRH Antagonist Treatment after Sublethal Total Body Irradiation (SL-TBI) Accelerates Lymphoid Recovery

This example illustrates that SSI agents administered subsequent to a sublethal dose of irradiation accelerate recovery of the immune system. Injury of lymphoid compartments and the consequent lymphopenia are one of the major causes of morbidly and mortality not only in BMT recipients but also following events of radiological accidents. The identification of immune-regenerative strategies to mitigate deleterious radiation effects after accidental exposure or terrorist attacks represents an unmet clinical challenge.

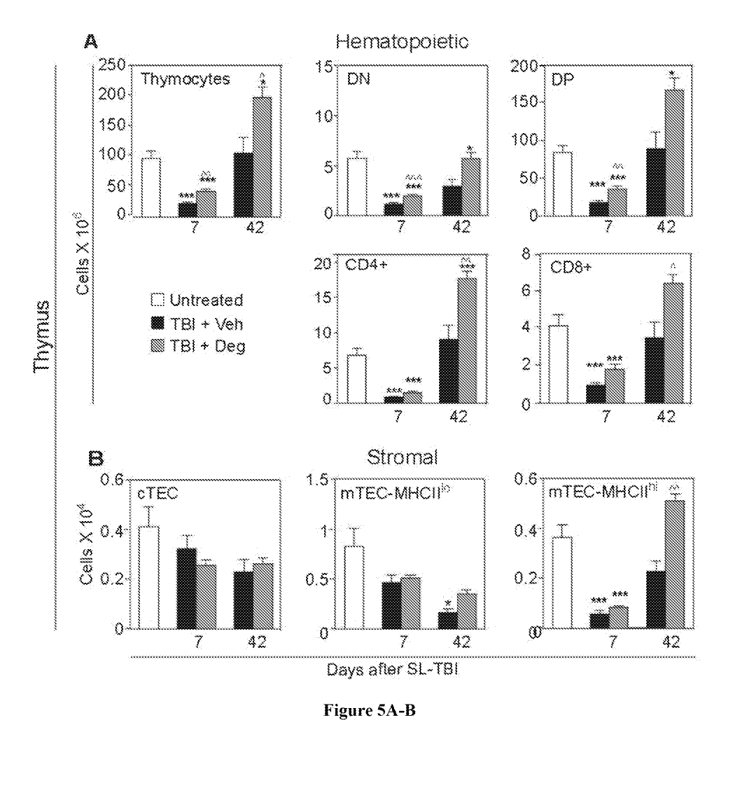

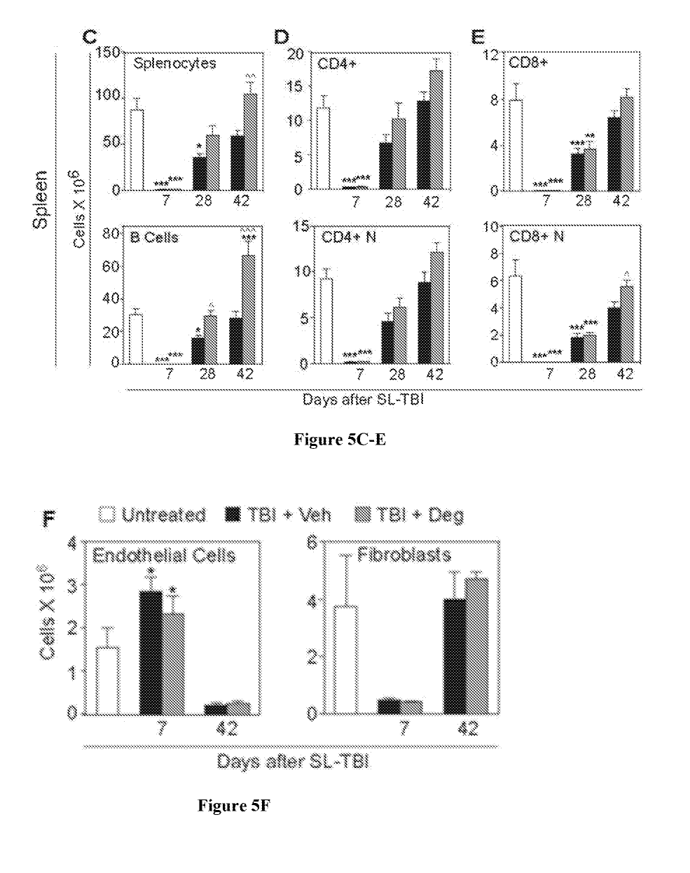

We investigated the effects on lymphoid reconstitution when Degarelix was administered 24 h after exposure of SL-TBI. We irradiated B6 male mice and injected them 24 h hours later with vehicle or Degarelix. Mice that have been treated with Degarelix had significantly increased thymus cellularity compared to vehicle treated control mice as early as day 7 after irradiation (FIG. 5A). Forty-two days after SL-TBI while the vehicle treated control returned to untreated levels the Degarelix treated mice showed once again significantly higher thymic cellularity compared to vehicle treated an untreated mice. Analyzing the thymocytes subsets we found a robust increase in thymopoiesis after Degarelix treatment manifested by significant increase in DP, correlated with a significant increased in the number of all DN subsets (FIG. 5A). Despite a trend toward increased CD4⁺ and CD8⁺ thymocytes counts, we did not detect a significant enhancement compared to irradiated control mice. In the Degarelix group, all thymic subsets were significantly increased 42 days after SL-TBI compared to vehicle-treated and untreated mice (FIG. 5A). Analysis of thymic stroma 7 days after SL-TBI did not show significant difference between vehicle and Degarelix treated mice (FIG. 5B). mTEC^{hi} cell number was significantly increased in Degarelix treated mice at day 42 after SL-TBI (FIG. 5B). mTEC^{lo}, fibroblast, and endothelial cells did not show differences between the irradiated groups (FIG. 5B and FIG. 5F).

We then investigated the effect of post-SL-TBI LHRH-Ant treatment in the peripheral reconstitution. Seven days after SL-TBI splenic cellularity was severity depleted in all groups. Starting from day 28, while vehicle treated mice still presented significant lower splenic cellularity compared to untreated mice, LHRH-Ant treated mice recovered faster from lymphoid depletion (FIG. 5C top). B cell counts were significantly increased in the LHRH-Ant group at day 28 and 42 compared to vehicle group (FIG. 5C bottom). Total numbers of CD4⁺ and CD8⁺ T cells and respectively subsets showed a consistent increasing trend in LHRH-Ant treated mice. CD8⁺naïve T cells were the most increased population 42 days after SL-TBI in LHRH-Ant treated mice (FIG. 5E). Our results suggest that LHRH antagonist treatment manifested regenerative effects on thymus and spleen when administered 24 h after SL-TBI.

### Example 8

### LHRH Antagonist Treatment after a Lethal Dose of Irradiation Promotes Hematopoietic Recovery

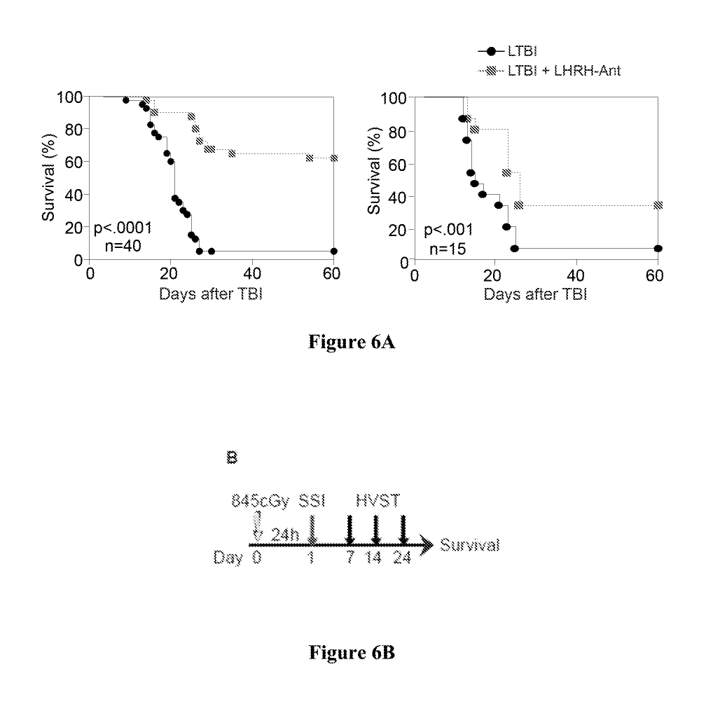

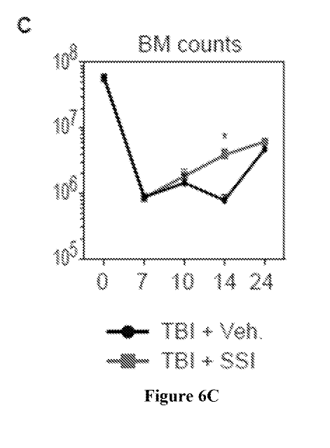

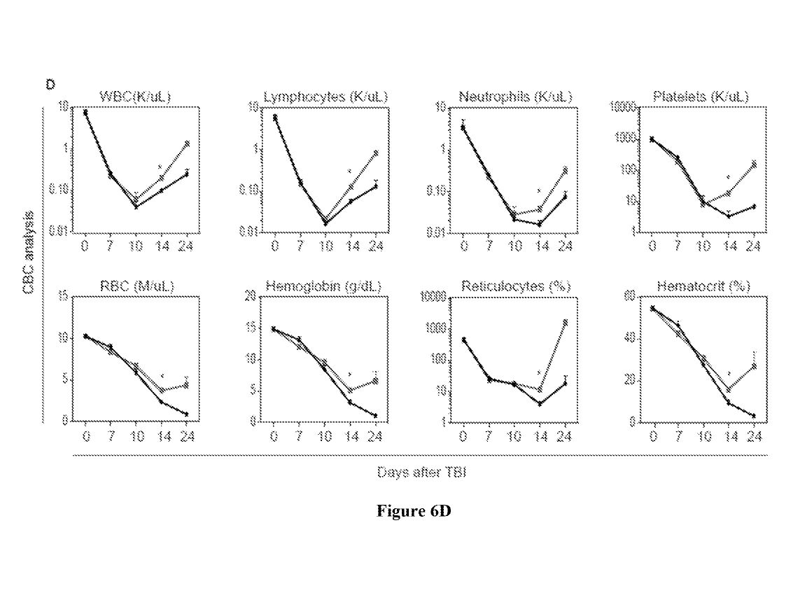

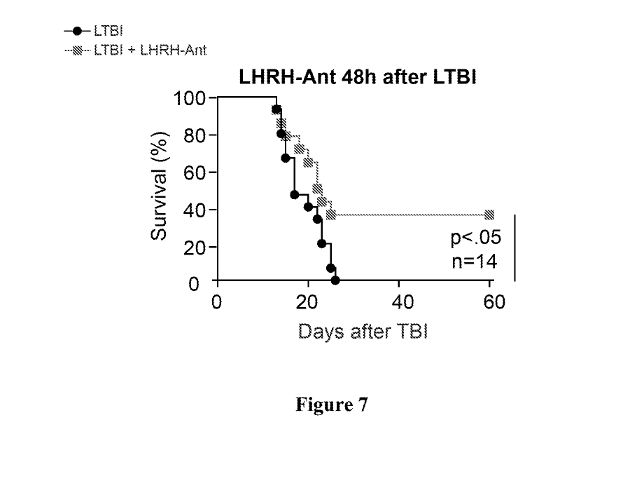

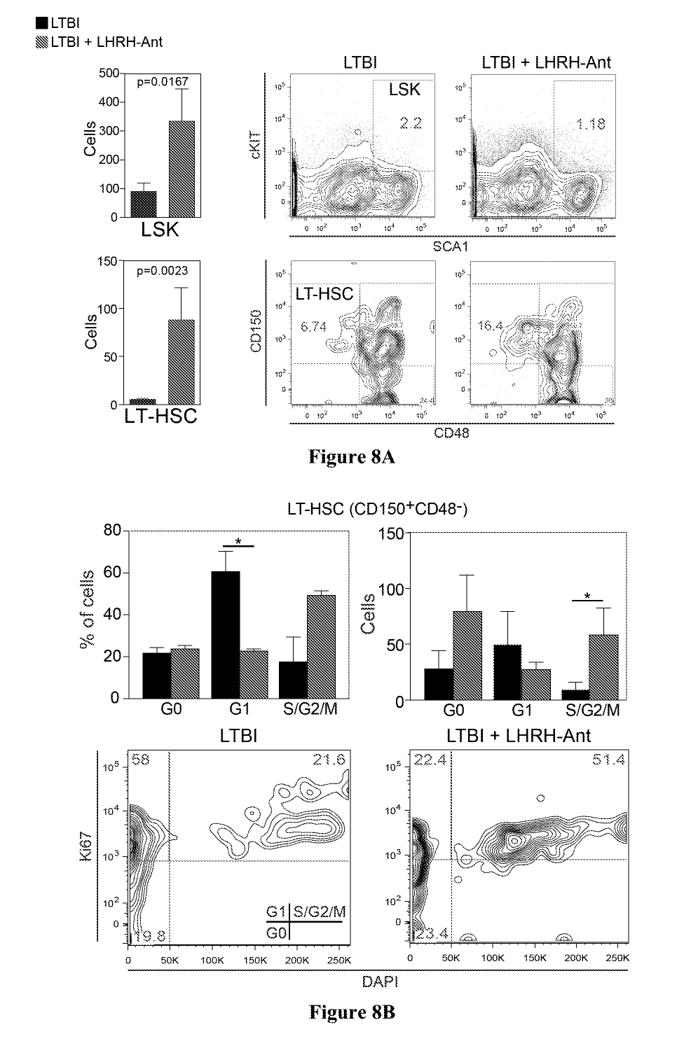

This example illustrates that SSI agents promote survival and hematopoietic recovery if administered subsequent to exposure to an otherwise lethal dose of radiation. Seven week-old male C57BL/6 mice were given a lethal dose of irradiation (845 cGy) and treated 24 (FIG. 6A) or 48 (FIG. 7) hours later with a single dose of vehicle (mannitol, circles) or LHRH antagonist (Degarelix, squares). Mouse survival was monitored daily. Data were analyzed using the Mantel-Cox log-rank test comparing Degarelix to vehicle alone. ****p<0.0001, n=40 (males); ***p<0.001, n=15 (females); *p<0.05, n=14 (males and females). FIG. 6B shows a schematic of experiment protocol for comprehensive analysis of hematopoietic reconstitution after lethal radiation injury. Total BM cellularity at days 7, 10, 14 and 24 after lethal TBI is shown in FIG. 6C. Complete blood count (CBC) of peripheral blood at days 7, 10, 14 and 24 after lethal TBI in mice treated with sex steroid inhibition (Degarelix) or vehicle alone (mannitol) is shown in FIG. 6D. At day 24, mannitol group (n=2), all other treatments and time points (n=5). Recovery of hematopoietic stem and progenitor lineages (Sca1⁺ckit⁺ and CD150⁺CD48⁻) after treatment with an LHRH antagonist or vehicle is shown in FIG. 8.

As shown in FIGS. 6A and 7, the window for treatment extends to at least 48 hours after total body irradiation (TBI). Further, after treatment with Degarelix, there is significantly more total Lineage-Sca1⁺ckit⁺ (LSK) as well as long-term hematopoietic stem cells (LT-HSC) in Degarelix-treated animals as compared to controls. Further, we observed an induction of LT-HSCs to proliferate (FIG. 8B), which is a prerequisite to reconstitution of hematopoiesis.

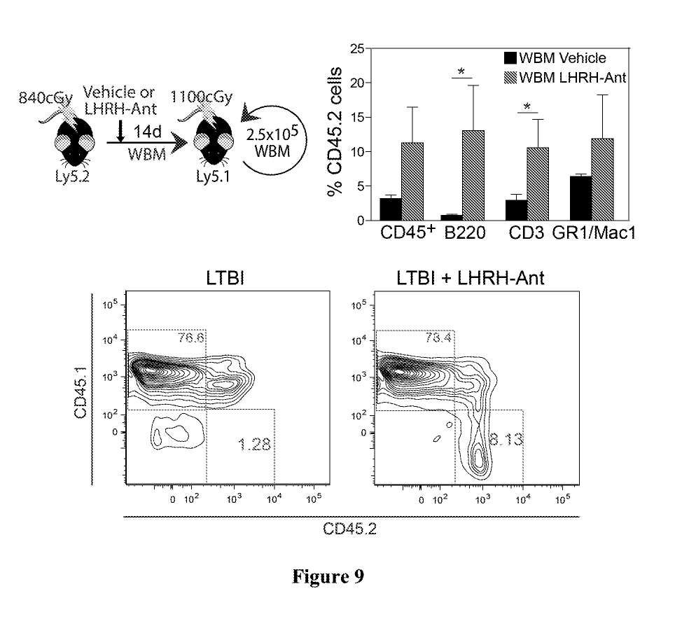

In a similar experiment, we isolated CD45.2⁺ bone marrow and spleen cells from mice that had been treated with Degarelix (and lethal TBI) and transferred them into lethally irradiated recipients (along with supporting CD45.1⁺ bone marrow cells to ensure survival). We could detect cells in the recipients that were derived from the CD45.2⁺ donor cells (FIG. 9). Therefore, these data demonstrate that there are more stem cells in the Degarelix-treated group than the control group, which further confirms why there is a higher incidence of survival in the Degarelix-treated group as compared to the control group.

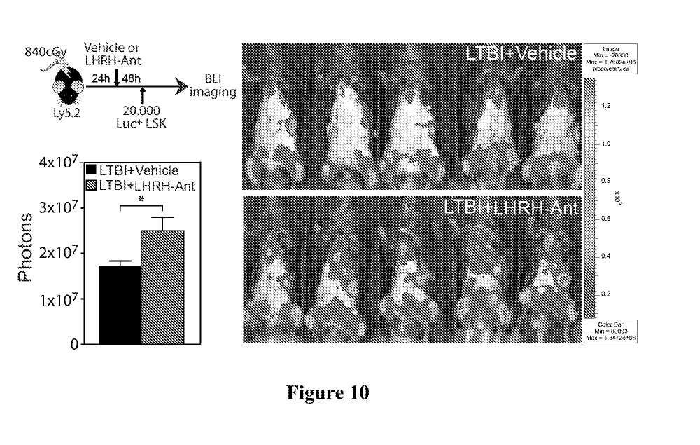

We continued our analysis of the increase in hematopoietic stem cells (HSCs) in mice treated with a LHRH antagonist after irradiation using a reporter molecule (Luciferase). We administered a lethal dose of irradiation (840 cGy) to groups of mice and treated them 24 hours later with a single dose of vehicle (LTBI, black) or Degarelix (LTBI+LHRH-Ant, grey). At 48 hours after treatment, 20,000 Luciferase-positive Lineage-Sca1⁺ckit⁺ (Luc⁺ LSK) hematopoietic stem cells were transferred to animals in each treatment group. Degarelix-treated animals had consistently more expansion of Luciferase-positive HSCs after treatment as compared to vehicle-treated animals (control), which is consistent with the increase in the number of HSCs in the same animals (FIG. 10). Therefore, these data confirm that sex steroid ablation increases the expansion of hematopoietic stem cells after radiation injury.

### Example 9

### Sex Steroid Ablation (SSA)-Mediated Regenerative Effects May be Androgen-Independent

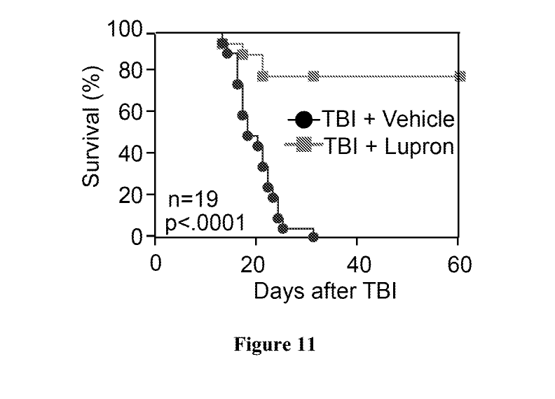

This example further illustrates that SSI agents promote survival and hematopoietic recovery if administered subsequent to exposure to an otherwise lethal dose of radiation. This example also indicates that sex steroid ablation (SSA)-mediated regenerative effects of SSI agents described herein may be androgen-independent. We wanted to determine whether LHRH agonists confer a similar survival benefit to irradiated mice as LHRH antagonists. As described above, mice were given a lethal dose of irradiation (845 cGy) and treated 24 hours later with a single dose of vehicle (PBS, circles) or LHRH agonist (Lupron [leuprolide], squares). Mouse survival was monitored daily (as described above). Lupron is an LHRH-receptor agonist and the standard of care for clinical sex steroid ablation. Since Lupron is a receptor agonist, Lupron first leads to a spike in sex steroid production before sensitization of the receptor, which leads to castrate levels of sex steroids. Thus, we reasoned that mice treated with Lupron would have either no effect on survival after lethal TBI or even worse survival. Surprisingly, we found that mice treated with Lupron also had significant survival when administered after lethal TBI (FIG. 11).

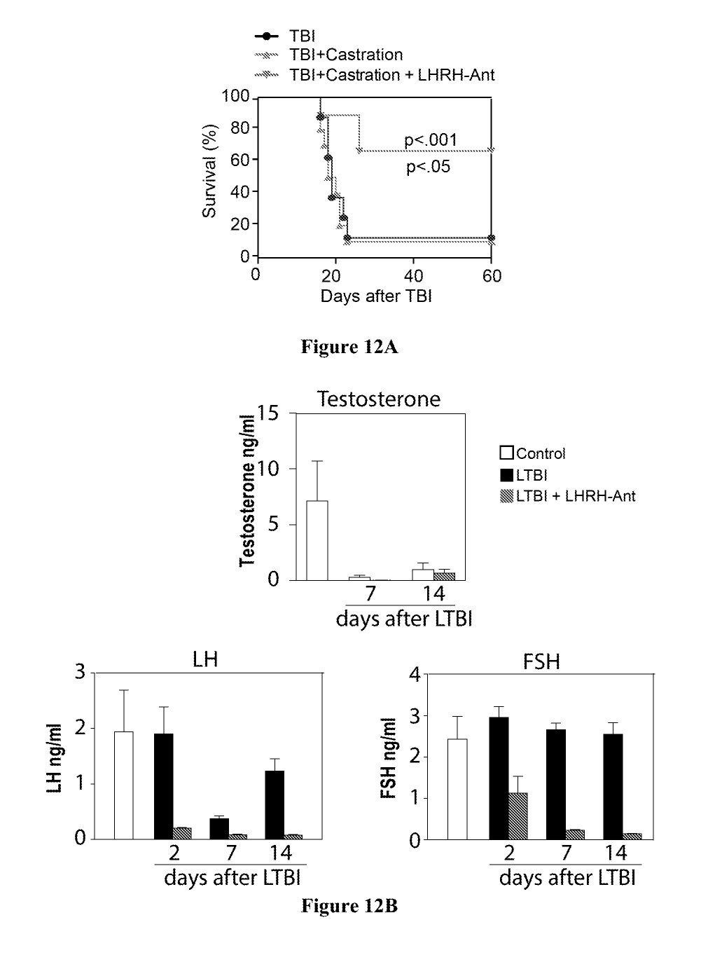

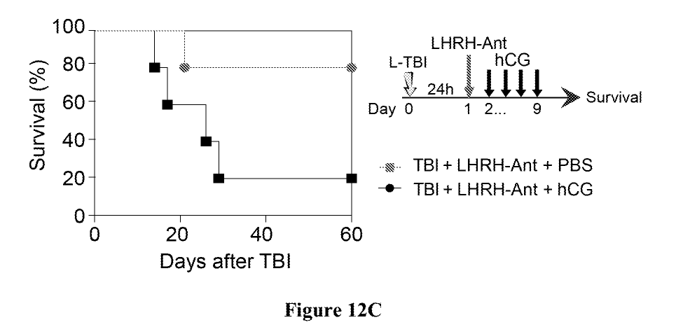

In another experiment, we wanted to determine if downstream and/or upstream targets could also have an effect on survival after TBI in mice. Mice were surgically castrated one day before total body irradiation (TBI) and administered vehicle (control) or Degarelix (LHRH-Ant). Mouse survival was monitored daily (as described above). Measurements of testosterone, follicle-stimulating hormone (FSH) and luteinizing hormone (LH) were taken for each treatment group at several days post irradiation. Interestingly, we observed no change in survival as compared to control mice, however, surgically castrated mice that were also administered Degarelix (LHRH-Ant) demonstrated enhanced survival (FIG. 12A). Further, we also observed that the level of testosterone was drastically less for both treatment groups as compared to controls, whereas the LH and FSH levels seemed relatively unchanged (FIG. 12B). Therefore, the maintenance of the survival benefit in Degarelix-treated (LHRH-Ant) mice even after castration suggests that the ablation of sex steroids does not lead to a survival benefit, but is achieved rather by some other mechanism.

In yet another experiment, mice were irradiated and administered Degarelix and a luteinizing hormone analogue (human chorionic gonadotropin, hCG). Consistent with our hypothesis, hematopoietic stem cells express the LH receptor and LT-HSCs express the LH receptor even more highly. We found that mice administered Degarelix in addition to hCG demonstrated significantly worse survival as compared to mice administered Degarelix alone (FIG. 12C).

Taken together, these data confirm that SSI agents (agonists or antagonists) promote survival and hematopoietic recovery if administered subsequent to exposure of an otherwise lethal dose of radiation and indicate that SSA-mediated regenerative effects may be androgen-independent.

### Materials and Methods for Examples

Mice and Bone Marrow Transplantation

C57BL/6 (H-2b) and B10.BR (H-2k), mice (The Jackson Laboratory) were used between 8 and 12 weeks of age for experiments with young mice, and were 9 months old for experiments with middle-aged mice. To model thymic damage and lymphoid depletion, C57BL/6 received SL-TBI with no hematopoietic rescue. All SL-TBI experiments were performed with a Cs-137γ-radiation source. The HSCT procedure was performed as previously described (Goldberg et al., 2009), with 1100 cGy split-dosed lethal irradiation of C57BL/6 hosts receiving 5×10⁶ T cell-depleted MHC-mismatched B10.BR BM cells. BM cells were T cell depleted by incubation with anti-Thy-1.2 for 40 min at 4° C. and incubation with LOW-TOX-M rabbit complement (Cedarlane Laboratories) for 40 min at 37° C. Cells were transplanted by tail vein infusion (0.2 ml total volume) into lethally irradiated recipients (C57BL/6) on day 0. To model GVHD, donor splenic T cells (5×106 B10.BR) were enriched using MILTENYI MACS CD5 purification (routine purity >90% purity). Recipient mice were monitored weekly for survival and clinical GVHD symptoms as previously described (Goldberg et al., 2009). All animal protocols were approved by the Memorial Sloan-Kettering Cancer Center Institutional Animal Care and Use Committee.

Reagents

Degarelix (as acetate), a third generation LHRH-Ant (Firmagon), was resuspended in sterile water for injection and administered S.C. to mice at a dose of 40 ug/g. Lupron (11.25 mg 3 month depot), an LHRH-Ag, was prepared according to the manufacturer's instructions and administrated I.M. to mice at a dose of 20 ug/g. Degarelix and Lupron were purchased from the Memorial Sloan-Kettering Cancer Center Pharmacy. Testosterone propionate (Sigma-Aldrich) was resuspended in peanut oil and injected daily S.C. (1 mg/mouse) in 100 ul. Surface antibodies against CD44 (IM7), EpCAM (G8.8), PDGFRa (APA5), PECAM-1 (390), CD45 (30-F11), H-2Kk (AF3-12.1.3) were purchased from eBioscience; anti-Ly-51 (BP-1), CD34 (RAM34), CD62L (MEL-14), H-2Kb (AF6-88.5), IFNγ (XMG1.2), IL-2 (JES6-5H4), c-Kit (2B8), CD3ε (145-2C11), CD25 (PC61), TER-119 (TER-110), CD8α (53-6.7), were purchased from BD Biosciences; anti-CD4 (RM4-5) and B220 (RA3-6B2) were purchased form Invitrogen; anti-CD44 (IM7), CD90.2 (30-H12) and IA/IE (M5/114.15.2) were purchased from Biolegend; Ulex europaeus agglutinin 1 (UEA-1) was purchased from Vector Laboratories (Burlingame, Calif.). Flow cytometric analysis was performed on an LSRII (BD Biosciences) using FACSDIVA (BD Biosciences) or FLOWJO (Treestar Software).

Cell Isolation

Individual or pooled single cell suspensions of freshly dissected thymi were obtained by either mechanical dissociation or enzymatic digestion, as previously described (Gray et al., 2008). CD45− cells for quantitative PCR experiments were enriched by magnetic bead separation using an AUTOMACS (Miltenyi Biotech) or MACS separation LD columns (Miltenyi Biotech).

Cell Culture

cTEC cell line C9 cells were maintained in culture in DME supplemented with 10% fetal calf serum (FCS), 100 U/ml penicillin and 100 μg/ml streptomycin. For experiments with Dihydrotestosterone-2,3,4-¹³C₃ (DTH) solution (Sigma-Aldrich) and MDV3100 (Enzalutamide, from Selleckbio), cells were maintained in DME supplemented with 10% charcoal:dextran stripped fetal calf serum (Gemini Bioproducts). MDV3100 was reconstituted in DMSO and used in culture at the final concentration of 10 μM. For experiments with DTH and MDV3100, cells were pre-treated with MDV3100 30 minutes before DTH treatment. Splenocytes for in vitro studies were cultured in RPMI supplemented with 10% FCS, 2 mM L16 glutamine, 1 mM sodium pyruvate, 50 μM 2-mercaptoethanol, 100 U/ml penicillin and 100 ug/ml streptomycin.

T Cell In Vitro Assay

To evaluate T cell proliferation and cytokine production, spleens were harvested 42 days after SL-TBI and T cells were purified by CD5⁺ selection. Half of the cells were stimulated for 5 hours with PMA, Ionomycin (50 ng/ml and 1 ug/ml, respectively) and BD Golgi Plug (1 μl/ml) and cytokines evaluated by intracellular flow cytometric analysis. The remaining cells were CFSE (Invitrogen) labeled and plated on αCD3/αCD28 (5 μg and 1 μg, respectively) pre-coated plates. Proliferation was assessed by measuring the number of cell divisions 2 days after stimulation by flow cytometric analysis.

Real Time PCR

Reverse transcription-PCR was performed with QUANTITECT reverse transcription kit (QIAGEN). For real-time PCR, specific primer and probe sets were obtained from Applied Biosystems as follows: β-actin (Mm01205647_g1); Ccl25 (Mm00436443_m1); Cxcl12 (Mm00445553_m1); Dll1 (Mm01279269_m1); Dll4 (Mm00444619_m1); Foxn1 (Mm00433946_m1); Ill5 (Mm00434210_m1); Il8β (Mm00434225_m1); Il1β (Mm00434228_m1); Il7 (Mm01295803_m1); Kgf (Mm00433291_m1); Scf (Mm00442972_m1). PCR was done on ABI 7500 (Applied Biosystems) or Step-One Plus (Applied Biosystems) with TAQMAN UNIVERSAL PCR MASTER MIX (Applied Biosystems). Relative amounts of mRNA were calculated by the comparative ΔCt method.

ChIP

ChIP was performed using ChIP assay kit (Millipore) following manufacturer's instructions. Briefly, cTEC C9 were stimulated for 2 h with DTH, with or without pretreatment for 30 minutes with MDV3100. Cells were then crosslinked with formaldehyde for 10 minutes and then incubated for 5 minutes with glycine to block crosslinking. Cells were than scraped and resuspended in SDS lysis buffer for 10 minutes then sonicated using 30% amplitude (Branson Digital Sonifier) for 20 minutes on/60 minutes off for a total of 10 cycles. The immunoprecipitation was performed using 2 μg anti-AR or non-immune rabbit IgG as a negative control. After elution, the samples were deproteinated, and quantitative PCR were used to evaluate the result. The sequences of the primers against the mouse Dll4 promoter regions used for CHIP were: Region A forward 5′-ACCCCTTAGAGTTTCCACCC-3′ (SEQ ID NO: 2), reverse 5′-TCTTCCAACTTCTGGGCTTCC-3′ (SEQ ID NO: 3); Region B forward 5′-CCCACCTCTCTTTCGAACCT-3′ (SEQ ID NO: 4), reverse 5′-GTAGGCGTGTCACCTCAAGC-3′ (SEQ ID NO: 5); Region C forward 5′-GGCACTCCAGGCAGGTCTAC-3′ (SEQ ID NO: 6), reverse 5′-GTGGGGAACCGAGGTGAG-3′ (SEQ ID NO: 7); Region D forward 5′-CGATTTATTGACCGGCAGG-3′ (SEQ ID NO: 8), reverse 5′-CCGCATTTAGGAGTGAACCG-3′ (SEQ ID NO: 9). The relative amounts of immunoprecipitated DNA fragments were expressed as fold increased over the IgG control using the ΔCt method.

Identification of Transcription-Factor-Binding Sites (TFBS)

Whole genome rVISTA (Zambon et al., 2005) at a stringency of p<0.005 was used to predict potential AR binding sites 5000 bp upstream of the TSS. Putative TFBS were then further characterized using JASAR database (Bryne et al., 2008).

LCMV Challenge

Mice were challenged I.P. with 2×10⁵ LCMV-Armstrong PFUs 14 days after SL-TBI. PFU assays were performed as previously described (Ahmed et al., 1984). Briefly, 7.5×10⁵ Vero cells were plated in a 6-well plate on day −1 of assay. On day 8 after infection, mice were sacrificed: spleens were harvested and sonicated in 1 ml of RPMI using 30% amplitude (Branson Digital Sonifier) for 15″-20″ in ice. 0.2 ml of sonicate were plated in serial dilution (10-1 through 10-6) and covered with a 1:1 complete Medium 199:1% agarose mixture following 60 minutes of adsorption. Plates were incubated at 37° C. and after 4 days, additional 1:1 complete 199 medium (1% agarose containing neutral red dye) was added to wells. The following day, the number of plaques was assessed.

Statistics

Bars and error bars represent the mean+SEM for the various groups. Statistical analysis between two groups was performed with the nonparametric, unpaired Mann-Whitney U test or Student's t test for qPCR experiments. ANOVA was used for comparisons between more than two groups. Survival data were analyzed with the Mantel-Cox log-rank test. All experiments were performed at least twice with at least six mice per group. All statistics were calculated and display graphs generated using GraphPad Prism.

### EQUIVALENTS

Having thus described several aspects of at least one embodiment of this invention, it is to be appreciated by those skilled in the art that various alterations, modifications, and improvements will readily occur to those skilled in the art. Such alterations, modifications, and improvements are intended to be part of this disclosure, and are intended to be within the spirit and scope of the invention. Accordingly, the foregoing description and drawing are by way of example only and the invention is described in detail by the claims that follow.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context.

The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification.

Those skilled in the art will appreciate typical standards of deviation or error attributable to values obtained in assays or other processes described herein.

### REFERENCES

Ahmed, R., A. Salmi, L. D. Butler, J. M. Chiller, and M. B. Oldstone. 1984. Selection of genetic variants of lymphocytic choriomeningitis virus in spleens of persistently infected mice. Role in suppression of cytotoxic T lymphocyte response and viral persistence. J Exp Med 160:521-540.
Bryne, J. C., E. Valen, M. H. Tang, T. Marstrand, O. Winther, I. da Piedade, A. Krogh, B. Lenhard, and A. Sandelin. 2008. JASPAR, the open access database of transcription factor-binding profiles: new content and tools in the 2008 update. Nucleic acids research 36:D102-106.
Dudakov, J. A., G. L. Goldberg, J. J. Reiseger, A. P. Chidgey, and R. L. Boyd. 2009a. Withdrawal of Sex Steroids Reverses Age- and Chemotherapy-Related Defects in Bone Marrow Lymphopoiesis. Journal of immunology 182:6247-6260.
Dudakov, J. A., G. L. Goldberg, J. J. Reiseger, K. Vlahos, A. P. Chidgey, and R. L. Boyd. 2009b. Sex Steroid Ablation Enhances Hematopoietic Recovery following Cytotoxic Antineoplastic Therapy in Aged Mice. Journal of immunology 183:7084-7094.
Edgar, J. D. 2008. T cell immunodeficiency. Journal of clinical pathology 61:988-993. Goldberg, G. L., O. Alpdogan, S. J. Muriglan, M. V. Hammett, M. K. Milton, J. M. Eng, V. M. Hubbard, A. Kochman, L. M. Willis, A. S. Greenberg, K. H. Tjoe, J. S. Sutherland, A. Chidgey, M. R. van den Brink, and R. L. Boyd. 2007. Enhanced immune reconstitution by sex steroid ablation following allogeneic hemopoietic stem cell transplantation. J Immunol 178:7473-7484.
Goldberg, G. L., J. A. Dudakov, J. J. Reiseger, N. Seach, T. Ueno, K. Vlahos, M. V. Hammett, L. F. Young, T. S. P. Heng, R. L. Boyd, and A. P. Chidgey. 2010. Sex Steroid Ablation Enhances Immune Reconstitution Following Cytotoxic Antineoplastic Therapy in Young Mice. Journal of Immunology 184:6014-6024.
Goldberg, G. L., C. G. King, R. A. Nejat, D. Y. Suh, O. M. Smith, J. C. Bretz, R. M. Samstein, J. A. Dudakov, A. P. Chidgey, S. Chen-Kiang, R. L. Boyd, and M. R. van den Brink. 2009. Luteinizing hormone-releasing hormone enhances T cell recovery following allogeneic bone marrow transplantation. J Immunol 182:5846-5854.
Gray, D. H., A. L. Fletcher, M. Hammett, N. Seach, T. Ueno, L. F. Young, J. Barbuto, R. L. Boyd, and A. P. Chidgey. 2008. Unbiased analysis, enrichment and purification of thymic stromal cells. Journal of immunological methods 329:56-66.
Heng, T. S., G. L. Goldberg, D. H. Gray, J. S. Sutherland, A. P. Chidgey, and R. L. Boyd. 2005. Effects of castration on thymocyte development in two different models of thymic involution. J Immunol 175:2982-2993.
Heng, T. S., J. J. Reiseger, A. L. Fletcher, G. R. Leggatt, O. J. White, K. Vlahos, I. H. Frazer, S. J. Turner, and R. L. Boyd. 2012. Impact of sex steroid ablation on viral, tumour and vaccine responses in aged mice. PLoS One 7:e42677.
Hozumi, K., C. Mailhos, N. Negishi, K. Hirano, T. Yahata, K. Ando, S. Zuklys, G. A. Hollander, D. T. Shima, and S. Habu. 2008. Delta-like 4 is indispensable in thymic environment specific for T cell development. J Exp Med 205:2507-2513.
Itoi, M., N. Tsukamoto, and T. Amagai. 2007. Expression of Dll4 and CCL25 in Foxn1-negative epithelial cells in the post-natal thymus. Int Immunol 19:127-132.
Koch, U., E. Fiorini, R. Benedito, V. Besseyrias, K. Schuster-Gossler, M. Pierces, N. R. Manley, A. Duarte, H. R. Macdonald, and F. Radtke. 2008. Delta-like 4 is the essential, nonredundant ligand for Notch1 during thymic T cell lineage commitment. J Exp Med 205:2515-2523. Lai, K. P., J. J. Lai, P. Chang, S. Altuwaijri, J. W. Hsu, K. H. Chuang, C. R. Shyr, S. Yeh, and C. Chang. 2013. Targeting thymic epithelia AR enhances T-cell reconstitution and bone marrow transplant grafting efficacy. Mol Endocrinol 27:25-37.
Loblaw, D. A., D. S. Mendelson, J. A. Talcott, K. S. Virgo, M. R. Somerfield, E. Ben-Josef, R. Middleton, H. Porterfield, S. A. Sharp, T. J. Smith, M. E. Taplin, N. J. Vogelzang, J. L. Wade, Jr., C. L. Bennett, and H. I. Scher. 2004. American Society of Clinical Oncology recommendations for the initial hormonal management of androgen-sensitive metastatic, recurrent, or progressive prostate cancer. Journal of clinical oncology: official journal of the American Society of Clinical Oncology 22:2927-2941.
Mohtashami, M., D. K. Shah, H. Nakase, K. Kianizad, H. T. Petrie, and J. C. Zuniga-Pflucker. 2010. Direct comparison of Dll1- and Dll4-mediated Notch activation levels shows differential lymphomyeloid lineage commitment outcomes. J Immunol 185:867-876.
Olsen, N. J., G. Olson, S. M. Viselli, X. Gu, and W. J. Kovacs. 2001. Androgen receptors in thymic epithelium modulate thymus size and thymocyte development. Endocrinology 142:1278-1283.
Parkman, R., and K. I. Weinberg. 1997 Immunological reconstitution following bone marrow transplantation. Immunol Rev 157:73-78.
Roche, P. J., S. A. Hoare, and M. G. Parker. 1992. A consensus DNA-binding site for the androgen receptor. Mol Endocrinol 6:2229-2235.
Sambandam, A., I. Maillard, V. P. Zediak, L. Xu, R. M. Gerstein, J. C. Aster, W. S. Pear, and A. Bhandoola. 2005. Notch signaling controls the generation and differentiation of early T lineage progenitors. Nat Immunol 6:663-670.
Small, T. N., E. B. Papadopoulos, F. Boulad, P. Black, H. Castro-Malaspina, B. H. Childs, N. Collins, A. Gillio, D. George, A. Jakubowski, G. Heller, M. Fazzari, N. Kernan, S. MacKinnon, P. Szabolcs, J. W. Young, and R. J. O'Reilly. 1999. Comparison of immune reconstitution after unrelated and related T-cell-depleted bone marrow transplantation: effect of patient age and donor leukocyte infusions. Blood 93:467-480.
Storek, J., A. Joseph, G. Espino, M. A. Dawson, D. C. Douek, K. M. Sullivan, M. E. Flowers, P. Martin, G. Mathioudakis, R. A. Nash, R. Storb, F. R. Appelbaum, and D. G. Maloney. 2001. Immunity of patients surviving 20 to 30 years after allogeneic or syngeneic bone marrow transplantation. Blood 98:3505-3512.
Sutherland, J. S., G. L. Goldberg, M. V. Hammett, A. P. Uldrich, S. P. Berzins, T. S. Heng, B. R. Blazar, J. L. Millar, M. A. Malin, A. P. Chidgey, and R. L. Boyd. 2005. Activation of thymic regeneration in mice and humans following androgen blockade. J Immunol 175:2741-2753.
Sutherland, J. S., L. Spyroglou, J. L. Muirhead, T. S. Heng, A. Prieto-Hinojosa, H. M. Prince, A. P. Chidgey, A. P. Schwarer, and R. L. Boyd. 2008. Enhanced immune system regeneration in humans following allogeneic or autologous hemopoietic stem cell transplantation by temporary sex steroid blockade. Clinical cancer research: an official journal of the American Association for Cancer Research 14:1138-1149.
Takahama, Y. 2006. Journey through the thymus: stromal guides for T-cell development and selection. Nat Rev Immunol 6:127-135.
Tan, J. B., I. Visan, J. S. Yuan, and C. J. Guidos. 2005. Requirement for Notch1 signals at sequential early stages of intrathymic T cell development. Nat Immunol 6:671-679. Taub, D. D., and D. L. Longo. 2005. Insights into thymic aging and regeneration. Immunol Rev 205:72-93. van Poppel, H., and S. Nilsson. 2008. Testosterone surge: rationale for gonadotropinreleasing hormone blockers? Urology 71:1001-1006.
Williams, K. M., P. J. Lucas, C. V. Bare, J. Wang, Y. W. Chu, E. Tayler, V. Kapoor, and R. E. Gress. 2008. CCL25 increases thymopoiesis after androgen withdrawal. Blood 112:3255-3263. Wils, E. J., B. van der Holt, A. E. Broers, S. J. Posthumus-van Sluijs, J. W. Gratama, E. Braakman, and J. J. Cornelissen. 2011. Insufficient recovery of thymopoiesis predicts for opportunistic infections in allogeneic hematopoietic stem cell transplant recipients. Haematologica 96:1846-1854.
Zambon, A. C., L. Zhang, S. Minovitsky, J. R. Kanter, S. Prabhakar, N. Salomonis, K. Vranizan, I. Dubchak, B. R. Conklin, and P. A. Insel. 2005. Gene expression patterns define key transcriptional events in cell-cycle regulation by cAMP and protein kinase A. Proc Natl Acad Sci USA 102:8561-8566.
Zoller, A. L., and G. J. Kersh. 2006. Estrogen induces thymic atrophy by eliminating early thymic progenitors and inhibiting proliferation of beta-selected thymocytes. J Immunol 176:7371-7378.

We claim:

1. A method for treatment of radiation injury comprising the step of administering an agent that reduces the activity of a sex hormone to a subject suffering from or susceptible to radiation injury.

2. The method of claim 1, wherein the radiation injury comprises a reduction in the number of myeloid cells.

3. The method of claim 1, wherein the radiation injury comprises a reduction in circulating levels of lymphoid cells.

4. The method of claim 1, wherein the radiation injury comprises a reduction in circulating levels of hemoglobin or hematocrit.

5. The method of claim 1, wherein the radiation injury comprises a reduction the number of red blood cells.

6. The method of claim 1, wherein the radiation injury results from exposure to a lethal dose of radiation.

7. The method of claim 1, wherein the radiation injury results from accidental exposure to ionizing radiation.

8. The method of claim 1, wherein the radiation injury results from exposure to ionizing radiation from a weapon.

9. The method of any one of claims 1-9, wherein the agent is administered contemporaneously with radiation exposure.

10. The method of any one of claims 1-9, wherein the agent is administered subsequent to radiation exposure.

11. The method of claim 10, wherein the agent is administered at least 24 hours subsequent to radiation exposure.

12. The method of claim 10, wherein the agent is administered at least 48 hours subsequent to radiation exposure.

13. The method of any one of claims 1-12, wherein the agent reduces the level of a sex hormone in circulation.

14. The method of any one of claims 1-12, wherein the agent inhibits the synthesis of a sex hormone.

15. The method of any one of claims 1-12, wherein the agent inhibits activity of a sex hormone receptor.

16. The method of any one of claims 1-12, wherein the agent modulates activity at a leutinizing hormone releasing hormone (LHRH) receptor.

17. The method of any one of claims 1-12, wherein the agent is a leutinizing hormone releasing hormone (LHRH) antagonist.

18. The method of any one of claims 1-12, wherein the agent is a leutinizing hormone releasing hormone (LHRH) agonist.

19. The method of claim 17, wherein the LHRH antagonist is degarelix.

20. The method of claim 18, wherein the LHRH agonist is leuprolide.

21. The method of any one of claims 1-12, wherein the agent is an androgen receptor antagonist.

22. The method of any one of claims 1-12, wherein the agent is an estrogen receptor antagonist.

23. The method of any one of claims 1-12, wherein the agent is an upstream regulator of sex hormone activity.

24. The method of claim 23, wherein the upstream regulator is leutinizing hormone (LH), follicle-stimulating hormone (FSH) or LHRH.

25. A method for treatment of radiation injury comprising the step of removing or ablating at least one of a testicle or ovary from a subject suffering from or susceptible to radiation injury.

26. A method for promoting hematopoietic recovery subsequent to radiation injury in a subject comprising the step of administering an agent that reduces the activity of a sex hormone.

27. The method of claim 26, wherein the hematopoietic recovery comprises the protection of hematopoietic stem cells.

28. The method of claim 26, wherein the hematopoietic recovery comprises the recovery of white blood cells.

29. The method of claim 28, wherein the white blood cells are lymphocytes.

30. The method of claim 28, wherein he white blood cells are myeloid cells.

31. The method of claim 26, wherein the hematopoietic recovery comprises an improvement in one or more complete blood count measures.

32. The method of claim 31, wherein the improvement in one or more complete blood count measures comprises an increase in hemoglobin level, an increase in hematocrit level, an increase in red blood cell number, and combinations thereof.

33. The method of claim 26, wherein the hematopoietic recovery comprises an increase in bone marrow cellularity.

34. The method of claim 26, wherein the radiation injury results from exposure to a lethal dose of radiation.

35. The method of claim 26, wherein the radiation injury results from accidental exposure to ionizing radiation.

36. The method of claim 26, wherein the radiation injury results from exposure to ionizing radiation from a weapon.

37. The method of claim 26, wherein the agent is administered at least 24 hours subsequent to radiation exposure.

38. The method of any one of claims 26-37, wherein the agent reduces the level of a sex hormone in circulation.

39. The method of any one of claims 26-37, wherein the agent inhibits the synthesis of a sex hormone.

40. The method of any one of claims 26-37, wherein the agent inhibits activity of a sex hormone receptor.

41. The method of any one of claims 26-37, wherein the agent modulates activity at a leutinizing hormone releasing hormone (LHRH) receptor.

42. The method of any one of claims 26-37, wherein the agent is a leutinizing hormone releasing hormone (LHRH) antagonist.

43. The method of any one of claims 26-37, wherein the agent is a leutinizing hormone releasing hormone (LHRH) agonist.

44. The method of claim 42, wherein the LHRH antagonist is degarelix.

45. The method of claim 43, wherein the LHRH agonist is leuprolide.

46. The method of any one of claims 26-37, wherein the agent is an androgen receptor antagonist.

47. The method of any one of claims 26-37, wherein the agent is an estrogen receptor antagonist.

48. The method of any one of claims 26-37, wherein the agent is an upstream regulator of sex hormone activity.

49. The method of claim 48, wherein the upstream regulator is leutinizing hormone (LH), follicle-stimulating hormone (FSH) or LHRH.

50. A pharmaceutical composition for use in the treatment of radiation injury comprising an agent that reduces the activity of a sex hormone and a pharmaceutically acceptable carrier.

51. The pharmaceutical composition of claim 50, wherein the agent reduces the level of a sex hormone in circulation.

52. The pharmaceutical composition of claim 50, wherein the agent inhibits the synthesis of a sex hormone.

53. The pharmaceutical composition of claim 50, wherein the agent inhibits activity of a sex hormone receptor.

54. The pharmaceutical composition of claim 50, wherein the agent modulates activity at a leutinizing hormone releasing hormone (LHRH) receptor.

55. The pharmaceutical composition of claim 50, wherein the agent is a leutinizing hormone releasing hormone (LHRH) antagonist.

56. The pharmaceutical composition of claim 50, wherein the agent is a leutinizing hormone releasing hormone (LHRH) agonist.

57. The pharmaceutical composition of claim 55, wherein the LHRH antagonist is degarelix.

58. The pharmaceutical composition of claim 56, wherein the LHRH agonist is leuprolide.

59. The pharmaceutical composition of claim 50, wherein the agent is an androgen receptor antagonist.

60. The pharmaceutical composition of claim 50, wherein the agent is an estrogen receptor antagonist.

61. The pharmaceutical composition of claim 50, wherein the agent is an upstream regulator of sex hormone activity.

62. The pharmaceutical composition of claim 61, wherein the upstream regulator is leutinizing hormone (LH), follicle-stimulating hormone (FSH) or LHRH.


SEQUENCE LISTING (17 sequences)

SEQ ID NO: 1 - DNA, 15 bp, Artificial Sequence
  Description of Artificial Sequence Synthetic oligonucleotide
ggwacannntgttct

SEQ ID NO: 2 - DNA, 20 bp, Artificial Sequence
  Description of Artificial Sequence Synthetic primer
accccttagagtttccaccc

SEQ ID NO: 3 - DNA, 21 bp, Artificial Sequence
  Description of Artificial Sequence Synthetic primer
tcttccaacttctgggcttcc

SEQ ID NO: 4 - DNA, 20 bp, Artificial Sequence
  Description of Artificial Sequence Synthetic primer
cccacctctctttcgaacct

SEQ ID NO: 5 - DNA, 20 bp, Artificial Sequence
  Description of Artificial Sequence Synthetic primer
gtaggcgtgtcacctcaagc

SEQ ID NO: 6 - DNA, 20 bp, Artificial Sequence
  Description of Artificial Sequence Synthetic primer
ggcactccaggcaggtctac

SEQ ID NO: 7 - DNA, 18 bp, Artificial Sequence
  Description of Artificial Sequence Synthetic primer
gtggggaaccgaggtgag

SEQ ID NO: 8 - DNA, 19 bp, Artificial Sequence
  Description of Artificial Sequence Synthetic primer
cgatttattgaccggcagg

SEQ ID NO: 9 - DNA, 20 bp, Artificial Sequence
  Description of Artificial Sequence Synthetic primer
ccgcatttaggagtgaaccg

SEQ ID NO: 10 - DNA, 22 bp, Unknown
  Description of Unknown DLL4 promoter oligonucleotide
ccgggcacgcgcagagctggaa

SEQ ID NO: 11 - DNA, 22 bp, Unknown
  Description of Unknown DLL4 promoter oligonucleotide
gaaggcacgcgctgggcacgcc

SEQ ID NO: 12 - DNA, 22 bp, Unknown
  Description of Unknown DLL4 promoter oligonucleotide
gctagtgcagcctgggcccatc

SEQ ID NO: 13 - DNA, 22 bp, Unknown
  Description of Unknown DLL4 promoter oligonucleotide
cctgggactcggcttgcccata

SEQ ID NO: 14 - DNA, 22 bp, Unknown
  Description of Unknown DLL4 promoter oligonucleotide
ggaggtacgccccgagctcccc

SEQ ID NO: 15 - DNA, 22 bp, Unknown
  Description of Unknown DLL4 promoter oligonucleotide
tgaggcacgcgccggcctcatt

SEQ ID NO: 16 - DNA, 22 bp, Unknown
  Description of Unknown DLL4 promoter oligonucleotide
gttggcacgcggagggccagcc

SEQ ID NO: 17 - DNA, 22 bp, Unknown
  Description of Unknown DLL4 promoter oligonucleotide
tattgaccggcaggtgcgagca